SEQUENCE LISTING (10 sequences)

SEQ ID NO: 1 - DNA, 169 bp, unidentified
  source 1..169: mol_type = other DNA
tgcgcacccttagcgagaggtttatcattaaggtcaacctctggatgttgtttcggcatcctgcattgaatctgagttactgtctgttttccttgttggaacggagagcatcgcctgatgctctccgagccaaccaggaaacccgttttttctgacgtaagggtgcgca

SEQ ID NO: 2 - DNA, 963 bp, synthetic construct
  source 1..963: mol_type = other DNA
atgaaatctgcagagtatctgaatacgttccgccttaggaatttgggcctccccgtgatgaacaatctccacgatatgagcaaggcgactcgaatatccgtggaaacgctgagactgctcatctatacagcagactttcggtacaggatctacacggtcgaaaagaaggggcctgagaaacgcatgcgaacaatttatcaacctagccgagagctcaaggcgttgcagggctgggttcttcgaaacatccttgacaaactctcatcatcaccctttagtattgggtttgaaaagcaccaaagcatccttaacaacgcgacgccacacataggtgccaatttcatattgaacatcgacttggaggatttttttccgagcctcacagccaataaagtgttcggtgtttttcacagtcttgggtacaatcgccttattagttccgttcttaccaagatttgttgttacaagaatctcttgccccagggagcacccagcagtccgaaattggcgaatttgatttgttccaagctcgattatcgaatacaagggtacgcgggcagccggggactcatctatacccgctacgcagacgatcttacgctgtctgcccaatcaatgaagaaggtcgtaaaggcgcgggatttcttgttttctatcatcccgtccgagggcttggtaattaattccaaaaagacttgtatctcaggaccacgatctcagcgaaaagtgacaggactcgtcatttctcaagaaaaagtcggtatagggagagagaagtataaggaaatccgcgcgaagatccaccacatattctgtggcaagagcagcgagatagaacacgtccgaggctggttgtccttcatactgagcgtggactcaaaaagccaccgccggttgatcacctatatttcaaaactggaaaagaaatatggaaagaacccactcaacaaagctaaaacatag

SEQ ID NO: 3 - DNA, 1761 bp, synthetic construct
  source 1..1761: mol_type = other DNA
atgacaaaaacttcaaagctggatgcgctgcgggcggctactagtagggaagatttggcgaagattctcgacataaagttggtgtttctgacaaacgtgttgtaccgcataggatccgacaaccagtatacgcaattcacaatacccaaaaagggtaaaggtgtccgcaccatcagcgcaccaacggaccgacttaaggatatacagaggaggatttgtgatcttcttagtgactgtagggatgaaatctttgcgattaggaagatctctaataattactcattcggcttcgaaagaggaaaatcaattatactcaatgcttacaagcatcgagggaagcaaattatattgaacatcgaccttaaggacttctttgagagctttaactttgggagagtccgggggtactttctctccaaccaggacttcttgttgaacccagttgtggcaacaacgttggcgaaggccgcctgctacaacgggactctgcctcaggggtccccatgttcccctattataagtaaccttatctgtaacattatggacatgcggctcgcaaagctcgccaagaagtacggctgcacttatagtcgatatgcggatgacattacgatcagcaccaataaaaataccttcccgttggagatggcgactgtgcagcctgaaggggttgtgctgggcaaagtgctcgtaaaggagattgaaaattcaggtttcgagattaacgattctaagactagattgacctacaaaacaagtaggcaagaagtcaccgggctgacggttaatcggattgtaaacattgatcggtgctactacaaaaagacgagggcgctggctcacgcattgtatcggacaggagaatataaggtcccagacgagaacggtgttctggtatctggagggcttgacaagttggagggtatgtttgggtttatcgaccaggtggataaattcaacaacattaaaaaaaagttgaataagcaacccgacagatatgttctgacaaatgccactttgcacggatttaagctcaaattgaacgccagggagaaagcctatagcaaattcatctactacaaattcttccacggtaatacttgtcccacgatcataacagagggtaagacggataggatttaccttaaagctgccctccatagcctcgagacaagttatcctgaactgtttcgggagaaaacagatagtaagaagaaggagataaatctgaatatttttaaaagcaatgagaagaccaagtatttcctggatctcagcggcggcacagcagacctcaagaaattcgtggaacgctacaaaaataactacgcttcctattacggcagcgtaccgaaacaaccggtgataatggtgcttgataacgacacaggcccgtcagacctgttgaactttttgagaaacaaagttaagagttgtccagatgatgtaacagaaatgcgcaagatgaagtacatacatgtgttttacaatctgtacatagttctgactcccctgtctccatctggagagcaaacgtctatggaggacctctttcctaaagatatattggacattaagatagatggcaagaaattcaataaaaacaatgacggtgactccaaaacagagtatgggaagcacatattctcaatgcgcgttgtacgagataaaaagaggaagatagatttcaaggcattttgctgtatcttcgatgctattaaggatattaaagaacattacaaactgatgttgaattcctag

SEQ ID NO: 4 - protein, 319 aa, unidentified
KSAEYLNTFRLRNLGLPVMNNLHDMSKATRISVETLRLLIYTADFRYRIYTVEKKGPEKRMRTIYQPSRELKALQGWVLRNILDKLSSSPFSIGFEKHQSILNNATPHIGANFILNIDLEDFFPSLTANKVFGVFHSLGYNRLISSVLTKICCYKNLLPQGAPSSPKLANLICSKLDYRIQGYAGSRGLIYTRYADDLTLSAQSMKKVVKARDFLFSIIPSEGLVINSKKTCISGPRSQRKVTGLVISQEKVGIGREKYKEIRAKIHHIFCGKSSEIEHVRGWLSFILSVDSKSHRRLITYISKLEKKYGKNPLNKAKT

SEQ ID NO: 5 - protein, 586 aa, unidentified
MTKTSKLDALRAATSREDLAKILDIKLVFLTNVLYRIGSDNQYTQFTIPKKGKGVRTISAPTDRLKDIQRRICDLLSDCRDEIFAIRKISNNYSFGFERGKSIILNAYKHRGKQIILNIDLKDFFESFNFGRVRGYFLSNQDFLLNPVVATTLAKAACYNGTLPQGSPCSPIISNLICNIMDMRLAKLAKKYGCTYSRYADDITISTNKNTFPLEMATVQPEGVVLGKVLVKEIENSGFEINDSKTRLTYKTSRQEVTGLTVNRIVNIDRCYYKKTRALAHALYRTGEYKVPDENGVLVSGGLDKLEGMFGFIDQVDKFNNIKKKLNKQPDRYVLTNATLHGFKLKLNAREKAYSKFIYYKFFHGNTCPTIITEGKTDRIYLKAALHSLETSYPELFREKTDSKKKEINLNIFKSNEKTKYFLDLSGGTADLKKFVERYKNNYASYYGSVPKQPVIMVLDNDTGPSDLLNFLRNKVKSCPDDVTEMRKMKYIHVFYNLYIVLTPLSPSGEQTSMEDLFPKDILDIKIDGKKFNKNNDGDSKTEYGKHIFSMRVVRDKKRKIDFKAFCCIFDAIKDIKEHYKLMLNS

SEQ ID NO: 6 - protein, 312 aa, unidentified
MSIDIETTLQKAYPDFDVLLKSRPATHYKVYKIPKRTIGYRIIAQPTPRVKAIQRDIIEILKQHTHIHDAATAYVDGKNILDNAKIHQSSVYLLKLDLVNFFNKITPELLFKALARQKVDISDTNKNLLKQFCFWNRTKRKNGALVLSVGAPSSPFISNIVMSSFDEEISSFCKENKISYSRYADDLTFSTNERDVLGLAHQKVKTTLIRFFGTRIIINNNKIVYSSKAHNRHVTGVTLTNNNKLSLGRERKRYITSLVFKFKEGKLSNVDINHLRGLIGFAYNIEPAFIERLEKKYGESTIKSIKKYSEGG

SEQ ID NO: 7 - protein, 311 aa, unidentified
MDILQHISDLLLTKKSEIISFSLTAPYRYKIYKIAKRNSDKKRTIAHPSKELKFIQREITEYLTDKLPVHECAFAYKKGSSIKTNAQVHLHTKYLLKMDFENFFPSITPRLFFSKLRLANIDLTADDKVLLENILFFKSKRNSNLRLSIGAPSSPLISNFVMYFWDIEVQEICSKIGVNYTRYADDLTFSTNNKDVLFDIPDMLENVLPKYSLGRIRINHEKTVFSSKGHNRHVTGITLTNDNKLSIGRERKRKISAMIHHFINGKLSTDECNKLVGLLAFAKNIEPSFYKSMVIKYGSDNIYKLQKQKDK

SEQ ID NO: 8 - DNA, 169 bp, synthetic construct; identical to SEQ ID NO: 1
  source 1..169: mol_type = other DNA

SEQ ID NO: 9 - DNA, 199 bp, unidentified
  source 1..199: mol_type = other DNA
tgataagattccgtatgcgcacccttagcgagaggtttatcattaaggtcaacctctggatgttgtttcggcatcctgcattgaatctgagttactgtctgttttccttgttggaacggagagcatcgcctgatgctctccgagccaaccaggaaacccgttttttctgacgtaagggtgcgcatacggaatcttatca

SEQ ID NO: 10 - DNA, 11 bp, synthetic construct
  source 1..11: mol_type = other DNA
ccccccccccc

(19) United States
(12) Patent Application Publication
(10) Pub. No.: US 2025/0223586 A1
(43) Pub. Date: Jul. 10, 2025

(54) METHODS OF ASSESSING ENGINEERED RETRON ACTIVITY, AND USES THEREOF

(71) Applicant: The J. David Gladstone Institutes, a testamentary trust established under the Will of J. David, San Francisco, CA (US)

(72) Inventor: Seth Shipman, San Francisco, CA (US)

(21) Appl. No.: 18/850,945

(22) PCT Filed: Mar. 24, 2023
(86) PCT No.: PCT/US2023/016262

§ 371 Date: Sep. 25, 2024

Related U.S. Application Data
(60) Provisional application No. 63/323,535, filed on Mar. 25, 2022.

(51) Int. Cl. C12N 15/10

(52) U.S. Cl. CPC C12N 15/1082; C12N 15/1068; C12N 15/1096

(57) ABSTRACT

Methods and compositions are described herein that are useful for analyzing and identifying retron systems with improved replication fidelity and efficiency. The methods and compositions described herein facilitate the engineering, quantification, and identification of retron elements for precise genome engineering.

### PRIORITY APPLICATION

This application claims the benefit of priority to U.S. Provisional Patent Application Ser. No. 63/323,535, filed Mar. 25, 2022, the content of which is incorporated herein by reference in its entirety.

### INCORPORATION BY REFERENCE OF SEQUENCE LISTING

A Sequence Listing is provided herewith as an xml file, “2319133.xml” created on Mar. 21, 2023, and having a size of 14,531 bytes. The content of the xml file is incorporated by reference herein in its entirety.

### BACKGROUND

Exogenous DNA can be introduced into cells as a template to edit the cell's genome—such DNA is typically synthesized or assembled in vitro and then delivered to target cells. However, delivery can be inefficient, and low abundance of template DNA can be one reason why precise editing typically occurs at a low rate. One tool to produce that DNA inside cells is a retron, a bacterial retroelement that can produce many copies of its DNA by its own reverse transcriptase. However, while retron DNAs can be synthesized in high amounts, not all retrons are reproduced efficiently or with fidelity. In addition, the process for generating and evaluating DNAs generated in vivo can also introduce errors. Hence, more efficient methods are needed to identify which retron systems are more useful for genome editing.

### SUMMARY

Methods and compositions are described herein that are useful for analyzing and identifying retron systems with improved replication fidelity and efficiency. The methods and compositions described herein facilitate the engineering, quantification, and identification of retron elements for precise genome engineering.

The methods can include: (a) debranching retron nucleic acids and generating reverse transcribed DNA (RT-DNA) from retron RNA in the retron nucleic acids; (b) extending 3′ ends of the RT-DNA with one type of deoxynucleotide triphosphate (dNTP) to generate 3′-end extended RT-DNA; (c) generating a second strand complement to the 3′-end extended RT-DNA using a DNA polymerase and a primer that binds to the 3′ extended ends of the RT-DNA to generate a double-stranded RT-DNA; (d) attaching adapters onto the 3′ ends of the double-stranded RT-DNA; (e) sequencing the double-stranded RT-DNA; and combinations thereof. A debranching enzyme can be used for the debranching single-stranded retron nucleic acids. A terminal deoxynucleotidyl transferase (TdT) can be used for the extending 3′ ends of the RT-DNA. In some cases, extending 3′ ends of the RT-DNA can be carried out in the absence of cobalt. In some cases, extending 3′ ends of the RT-DNA can be carried out at room temperature. Extending 3′ ends of the RT-DNA can be carried out, for example, for 30 seconds to 360 seconds. In some cases, extending 3′ ends of the RT-DNA can be carried out for about 120 seconds. Extending the 3′ ends of the RT-DNA can be terminated after 30 seconds to 360 seconds. In some cases, extending 3′ ends of the RT-DNA can be terminated after about 120 seconds. One type of deoxynucleotide triphosphate (dNTP) used for extending 3′ ends of the RT-DNA can be dATP, dCTP, dGTP, or dTTP. However, in some cases, the one type of deoxynucleotide triphosphate (dNTP) used can be dATP or dCTP. In some cases, the one type of deoxynucleotide triphosphate (dNTP) can be dATP.

Attaching adapters onto the 3′ ends of the double-stranded RT-DNA can be carried out using a ligase. For example, the ligase can be a T4 ligase or a ligase that can catalyze ligation between single-stranded DNA (ssDNA) and single-stranded RNA (ssRNA) substrates. The adapters employed can be oligonucleotides. In some cases, the adapters can be oligonucleotides terminated in guanine.

The second strand complement can be made using a DNA polymerase without 3′-5′ exonuclease.

The retron nucleic acids can be single-stranded. In some cases, the retron nucleic acids are a mix of single-stranded nucleic acids and double-stranded nucleic acids. For example, the retron nucleic acids can be oligonucleotides. In some cases, the retron nucleic acids are oligonucleotides that are amplified or expressed from expression vectors or expression cassettes. The retron nucleic acids can be one or more types of retron (e.g., Eco1, Eco2, Ec48, E67, Ec73, Ec78, EC83, EC86, EC107, Ec107, Mx65, Mx162, Sa163, Vc81, Vc95, Vc137, Vc96, Ne144, or a combination thereof).

The retron nucleic acids comprise at least one library of retron nucleic acids. For example, the retron nucleic acids can be one or more libraries of retron variants, retron mutants, engineered retrons, or a combination thereof.

The retron nucleic acids can include exogenous or heterologous RNA or DNA. For example, the retron nucleic acids can include exogenous or heterologous RNA or DNA such as one or more gRNAs, DNA templates (donor DNAs) for genetic repair, antigens, proteins, regulatory RNAs of interest, or a combination thereof.

### DESCRIPTION OF THE FIGURES

FIG. 1A-1D illustrate methods for faithfully and efficiently identifying the sequences of retrons generated in vivo. FIG. 1A is a schematic illustrating methods for preparing reverse transcribed retron DNA (RT-DNA) (SEQ ID NO: 10). Such RT-DNA can be analyzed by sequencing. FIG. 1B illustrates extending 3′ ends of RT-DNA using terminal deoxynucleotidyl transferase (TdT) with either dATP or dCTP over time. The reaction conditions and the timing of the reaction can be controlled to provide desired extension lengths. FIG. 1C illustrates sequenced ratios of RT-DNA oligos by the type of terminal base added. The RT-DNA oligos were physically mixed at equal ratios and the TdT was used for extension base with (+Co) or without cobalt. Replicates of different oligo sets are represented in different colored circles. FIG. 1D is a schematic diagram illustrating an alternate RT-DNA preparation based on use of CircLigase.

FIG. 2A-2F illustrate multiplexed sequencing of six different RT-DNAs generated from different retron ncRNAs. FIG. 2A illustrates that the RT-DNA portion of the Eco1 retron ncRNA was sequenced by the methods described herein. FIG. 2B illustrates that the RT-DNA portion of the Eco2 retron ncRNA was sequenced by the methods described herein.

FIG. 2C illustrates that the RT-DNA portion of the Eco4 retron ncRNA was sequenced by the methods described herein. FIG. 2D illustrates that the RT-DNA portion of the Eco6 retron ncRNA was sequenced by the methods described herein. FIG. 2E illustrates that the RT-DNA portions of the Eco9 retron ncRNA was sequenced by the methods described herein. FIG. 2F illustrates that the RT-DNA portion of the Sen2 retron ncRNA was sequenced by the methods described herein. A schematic of each of the indicated ncRNAs is positioned above the sequence positions detected by sequencing, where the a1/a2 primer region is in blue, the ncRNA-RNA portion is in pink, and the RT-DNA in brown.

FIG. 3A-3B illustrate quantification of multiple ncRNA/RT-DNAs. FIG. 3A is a schematic diagram illustrating ncRNA library generation and quantification. FIG. 3B is a schematic illustrating that a change (mutation) in ncRNA can be linked to a barcode in RT-DNA. The colors of the segments in loop identify retrons with different mutations linked to different bar codes.

FIG. 4A-4B graphically illustrates quantification of RT-DNA production for 2,158 different insertion, deletion, and base swap mutants relative to their position along a linear ncRNA. FIG. 4A is a schematic of ncRNA structure highlighting different regions in the colors of the symbols in FIG. 4B. FIG. 4B graphically illustrates the quantities of different ncRNA modifications on RT-DNA production, relative to wild-type (dotted line), based upon the position of the modification along the ncRNA. The modifications included insertions, deletions, and base swap variants. Each circle is a single variant, at the position indicated on the ncRNA, moving from 5′ to 3′. The schematic shown in FIG. 4A identifies the regions of the ncRNA by colors. Black line is a moving average of all variants by position over a five-base window.

FIG. 5A-5G illustrate evaluation of ncRNA/RT-DNA features as well as retron similarities and differences. FIG. 5A shows a schematic of Eco1 and Eco4 ncRNAs, illustrating a difference between them in the loop identified with positions 1-3. Both have a1/a2 and stem-loop regions that can be modified as described herein (the a1/a2 regions are labeled and the stem-loop regions are shown in blue). FIG. 5B illustrates the relative abundance of RT-DNA from Eco1 variants having modified loop bases at positions 1-3 of the loop shown in FIG. 5A. Deeper red shades indicate more RT-DNA production. As illustrated, use of thymine (uridine) nucleotides at position 2 of the Eco1 retron loop indicated in FIG. 5A ncRNA provides improved RT-DNA (donor DNA) production. FIG. 5C illustrates the relative abundance of RT-DNA from Eco4 variants having modified loop bases at the positions indicated in FIG. 5A. Deeper red shades indicate more RT-DNA production. As illustrated, use of thymine (uridine) nucleotides at position 2 in the loop indicated in FIG. 5A does not improve RT-DNA (donor DNA) production in the Eco4 ncRNA as much as it does for the Eco1 ncRNA. Eco4 is less vulnerable than Eco1 to sequence alterations in this loop. FIG. 5D graphically illustrates the relative RT-DNA abundance of each Eco1 stem length variant analyzed, where the RT-DNA abundance is shown as a percentage of wild-type abundance (dashed line). As illustrated, the RT-DNA abundance varies depending upon the length of the stem up to about stem length 15. FIG. 5E graphically illustrates the relative RT-DNA abundance of different Eco1 a1/a2 stem length variants of Eco1 as a percentage of wild-type abundance (dashed line). FIG. 5F graphically illustrates the relative RT-DNA abundance of each Eco4 stem length variant as a percentage of wild-type abundance (dashed line). FIG. 5G graphically illustrates the relative RT-DNA abundance of different Eco4 a1/a2 stem length variants as a percentage of wild-type abundance (dashed line).

FIG. 6 graphically illustrates abundance of a library of retron reverse transcriptase (RT) variants relative to wild type, where the RT mutants were linked to barcodes in the RT-DNA (above) for quantification by sequencing. Purple circles are mutations to alanine, while orange circles are RT mutations to a maximally dissimilar amino acid.

FIG. 7A-7I illustrates RT-DNA production in eukaryotic cells. FIG. 7A shows a schematic of the retron cassette for expression in yeast, with qPCR primers indicated by half arrows. FIG. 7B illustrates increased production of mutant Eco1 RT-DNA with a longer a1/a2 region in yeast. Enrichment of the Eco1 RT-DNA/plasmid template in yeast over the plasmid alone was detected by qPCR, with each construct shown relative to uninduced expression, using methods like those described in FIGS. 2B and 7B. Circles show each of three biological replicates, with black for the wild type a1/a2 length (12 nucleotides) and green for the extended a1/a2 (27 nucleotides). FIG. 7C illustrates increased production of mutant Eco2 RT-DNA with a longer a1/a2 region in yeast. Enrichment of the Eco2 RT-DNA/plasmid template over the plasmid alone was detected by qPCR, using methods like those described in FIGS. 2B and 7B. FIG. 7D shows a schematic of expression of retrons in mammalian cells, as detected by qPCR (primers indicated by half arrows). FIG. 7E illustrates enrichment in HEK293T cells of the Eco1 RT-DNA/plasmid template over the plasmid alone as detected by qPCR, using methods like those described in FIGS. 2B and 7B. FIG. 7F illustrates enrichment in HEK293T cells of the Eco2 RT-DNA/plasmid template over the plasmid alone as detected by qPCR, using methods like those described in FIGS. 2B and 7B. FIG. 7G shows a gel with size-separated Eco1 and Eco2 RT-DNA that had been isolated from yeast and subjected to PAGE analysis. The ladder is shown at a different exposure to the left of the gel image. FIG. 7H illustrates enrichment of Eco1 RT-DNA/plasmid template when uninduced compared to a dead RT construct. Closed circles show each of three biological replicates, with red for the dead RT version and black for the live RT. FIG. 7I lustrates enrichment of Eco1 RT-DNA/plasmid template in HEK293T cells, using methods like those described herein.

FIG. 8A-8B illustrate modified retrons that can be used in genome editing. FIG. 8A shows a schematic of an RT-DNA template for recombineering. The retron ncRNA was modified in the msd region (blue) to include a long loop (green) that contains homology to a bacterial genomic locus but has one or more nucleotide modifications (repair nucleotides; asterisks). FIG. 8B graphically illustrates fold enrichment of the Eco1-based recombineering RT-DNA/plasmid template over the plasmid alone in E. coli, as detected by qPCR, with each construct shown relative to uninduced. Circles show each of three biological replicates, with black for the wild type a1/a2 length and green for the extended a1/a2.

FIG. 9 illustrates retron features that can be modified.

### DETAILED DESCRIPTION

Described herein are methods and compositions useful for analyzing and identifying retron systems with improved replication fidelity and efficiency. Also described herein are methods for generating multitudes (e.g., thousands) of retron variants and testing them in pooled assays for the fidelity and levels of production of retron reverse transcribed DNA (RT-DNA). For example, the effect of modifying the retron ncRNA on RT-DNA production can be quantified by synthesizing retron ncRNA variants as oligonucleotide variant pools (e.g., in vitro). Such variant oligonucleotides can reside within or be subcloned into expression vectors (e.g., using efficient golden-gate style construction) to generate one or more libraries or sublibraries of different retron nucleic acids. The ncRNAs encoded in the libraries/sublibraries can be expressed in host cells along with a selected, matched reverse transcriptase, so that the reverse transcriptase can generate a multitude of different RT-DNAs in host cell types of interest.

The methods described herein can, for example, include: (a) debranching retron nucleic acids and generating reverse transcribed DNA (RT-DNA) from retron RNA in the retron nucleic acids; (b) extending 3′ ends of the RT-DNA with one type of deoxynucleotide triphosphate (dNTP) to generate 3′-end extended RT-DNA; (c) generating a second strand complement to the 3′-end extended RT-DNA using a DNA polymerase and a primer that binds to the 3′ extended ends of the RT-DNA to generate a double-stranded RT-DNA; (d) attaching adapters onto the 3′ ends of the double-stranded RT-DNA; and (e) sequencing the double-stranded RT-DNA. A debranching enzyme can be used for the debranching single-stranded retron nucleic acids.

The retron nucleic acids can be synthesized in vivo within host cells by reverse transcription and then isolated from the host cells. Different retron reverse transcriptases exist and they can vary in efficiency, depending to some extend on the host cell and the retron ncRNA template. Hence, the methods described herein can also be used to evaluate which reverse transcriptase can optimize retron DNA production from particular retron ncRNAs and within particular host cells. For example, the reverse transcriptase can be carried on a separate inducible plasmid or integrated into a genomic site within selected host cells. In yeast, for example, a reverse transcriptase can be integrated into the HIS3 locus and a galactose-inducible promoter can be used to induce expression of the reverse transcriptase. In another example, in human cells an inducible reverse transcriptase can be expressed upstream of the ncRNA and the expression of the reverse transcriptase can be driven by doxycycline.

The retron nucleic acids can be obtained from host cells that express a single type of retron ncRNA or from a population (library) of host cells, where the different cells in the population can express a few or a multitude of potentially different retron ncRNAs.

After generating a double-stranded RT-DNA, adapter sequences can be added to retron nucleic acids. For example, a pair of adapter sequences can be added at the 5′ and 3′ ends of each retron construct. For example, such adapters can be used for purification, subcloning, and/or preparation of the second strand of the RT-DNA. In some cases, the adaptors can be used for incorporation of variant retron nucleic acids into one or more types of expression cassettes or expression vectors.

### Retrons

The present disclosure provides methods for analyzing retron nucleic acids, including one or more retron variants, retron mutants, engineered retrons, or combinations thereof. For example, the retron nucleic acids can be modified to enhance production of retron reverse transcribed DNA in a host cell. The retron nucleic acids can also be modified to include useful exogenous or heterologous nucleic acids, thereby allowing in vivo production of substantial amounts of products such as gRNAs, templates for genomic repair (donor DNAs), coding regions for antigens, coding regions for proteins, coding regions for regulatory RNAs, and the like.

A retron is a distinct DNA sequence originally found in the genome of many bacteria species that codes for reverse transcriptase and a unique single-stranded DNA/RNA hybrid called multicopy single-stranded DNA (msDNA). Retron msr RNA is the non-coding RNA (ncRNA) produced by retron elements and is the immediate precursor to the synthesis of msDNA.

While msDNA and reverse transcribed DNA (RT-DNA) are related, the term reverse transcribed DNA (RT-DNA) is used herein to refer to any retron-related reverse transcribed DNA, whether modified or not, while the term msDNA refers to wild type, or natural retron msDNA.

The ncRNA includes a pre-msr sequence, an msr gene encoding multicopy single-stranded RNA (msRNA); an msd gene encoding multicopy single-stranded DNA (msDNA); a post-msd sequence and a ret gene encoding a reverse transcriptase. In nature, synthesis of retron DNA by the retron-encoded reverse transcriptase can provide a DNA/RNA chimeric product which is composed of single-stranded DNA encoded by the msd gene linked to single-stranded RNA encoded by the msr gene. The retron msr RNA contains a conserved guanosine residue at the end of a stem loop structure. A strand of the msr RNA is joined to the 5′ end of the msd single-stranded DNA by a 2′-5′ phosphodiester linkage at the 2′ position of this conserved guanosine residue.

For example, a wild type retron-Eco1 ncRNA (also called ec86 or retron-Eco1 ncRNA) can have the sequence shown below as SEQ ID NO: 1.

| | |
|---|---|
| 1 | TGCGCACCCT TAGCGAGAGG TTTATCATTA AGGTCAACCT |
| 41 | CTGGATGTTG TTTCGGCATC CTGCATTGAA TCTGAGTTAC |
| 81 | TGTCTGTTTT CCTTGTTGGA ACGGAGAGCA TCGCCTGATG |
| 121 | CTCTCCGAGC CAACCAGGAA ACCCGTTTTT TCTGACGTAA |
| 161 | GGGTGCGCA |

An example of an Eco1 human-codon optimized reverse transcriptase (RT) sequence that can be used is shown below as SEQ ID NO:2.

| | |
|---|---|
| 1 | ATGAAATCTG CAGAGTATCT GAATACGTTC CGCCTTAGGA |
| 41 | ATTTGGGCCT CCCCGTGATG AACAATCTCC ACGATATGAG |
| 81 | CAAGGCGACT CGAATATCCG TGGAAACGCT GAGACTGCTC |
| 121 | ATCTATACAG CAGACTTTCG GTACAGGATC TACACGGTCG |
| 161 | AAAAGAAGGG GCCTGAGAAA CGCATGCGAA CAATTTATCA |
| 201 | ACCTAGCCGA GAGCTCAAGG CGTTGCAGGG CTGGGTTCTT |
| 241 | CGAAACATCC TTGACAAACT CTCATCATCA CCCTTTAGTA |
| 281 | TTGGGTTTGA AAAGCACCAA AGCATCCTTA ACAACGCGAC |
| 321 | GCCACACATA GGTGCCAATT TCATATTGAA CATCGACTTG |
| 361 | GAGGATTTTT TTCCGAGCCT CACAGCCAAT AAAGTGTTCG |
| 401 | GTGTTTTTCA CAGTCTTGGG TACAATCGCC TTATTAGTTC |
| 411 | CGTTCTTACC AAGATTTGTT GTTACAAGAA TCTCTTGCCC |
| 481 | CAGGGAGCAC CCAGCAGTCC GAAATTGGCG AATTTGATTT |
| 521 | GTTCCAAGCT CGATTATCGA ATACAAGGGT ACGCGGGCAG |
| 561 | CCGGGGACTC ATCTATACCC GCTACGCAGA CGATCTTACG |
| 601 | CTGTCTGCCC AATCAATGAA GAAGGTCGTA AAGGCGCGGG |
| 641 | ATTTCTTGTT TTCTATCATC CCGTCCGAGG GCTTGGTAAT |
| 681 | TAATTCCAAA AAGACTTGTA TCTCAGGACC ACGATCTCAG |
| 721 | CGAAAAGTGA CAGGACTCGT CATTTCTCAA GAAAAAGTCG |
| 761 | GTATAGGGAG AGAGAAGTAT AAGGAAATCC GCGCGAAGAT |
| 801 | CCACCACATA TTCTGTGGCA AGAGCAGCGA GATAGAACAC |
| 841 | GTCCGAGGCT GGTTGTCCTT CATACTGAGC GTGGACTCAA |
| 881 | AAAGCCACCG CCGGTTGATC ACCTATATTT CAAAACTGGA |
| 921 | AAAGAAATAT GGAAAGAACC CACTCAACAA AGCTAAAACA |
| 961 | TAG |

An example of an Eco2 human-codon optimized reverse transcriptase (RT) sequence is shown below as SEQ ID NO:3.

| | |
|---|---|
| 1 | ATGACAAAAA CTTCAAAGCT GGATGCGCTG CGGGCGGCTA |
| 41 | CTAGTAGGGA AGATTTGGCG AAGATTCTCG ACATAAAGTT |
| 81 | GGTGTTTCTG ACAAACGTGT TGTACCGCAT AGGATCCGAC |
| 121 | AACCAGTATA CGCAATTCAC AATACCCAAA AAGGGTAAAG |
| 161 | GTGTCCGCAC CATCAGCGCA CCAACGGACC GACTTAAGGA |
| 201 | TATACAGAGG AGGATTTGTG ATCTTCTTAG TGACTGTAGG |
| 241 | GATGAAATCT TTGCGATTAG GAAGATCTCT AATAATTACT |
| 281 | CATTCGGCTT CGAAAGAGGA AAATCAATTA TACTCAATGC |
| 321 | TTACAAGCAT CGAGGGAAGC AAATTATATT GAACATCGAC |
| 361 | CTTAAGGACT TCTTTGAGAG CTTTAACTTT GGGAGAGTCC |
| 401 | GGGGGTACTT TCTCTCCAAC CAGGACTTCT TGTTGAACCC |
| 441 | AGTTGTGGCA ACAACGTTGG CGAAGGCCGC CTGCTACAAC |
| 481 | GGGACTCTGC CTCAGGGGTC CCCATGTTCC CCTATTATAA |
| 521 | GTAACCTTAT CTGTAACATT ATGGACATGC GGCTCGCAAA |
| 561 | GCTCGCCAAG AAGTACGGCT GCACTTATAG TCGATATGCG |
| 601 | GATGACATTA CGATCAGCAC CAATAAAAAT ACCTTCCCGT |
| 641 | TGGAGATGGC GACTGTGCAG CCTGAAGGGG TTGTGCTGGG |
| 681 | CAAAGTGCTC GTAAAGGAGA TTGAAAATTC AGGTTTCGAG |
| 721 | ATTAACGATT CTAAGACTAG ATTGACCTAC AAAACAAGTA |
| 761 | GGCAAGAAGT CACCGGGCTG ACGGTTAATC GGATTGTAAA |
| 801 | CATTGATCGG TGCTACTACA AAAAGACGAG GGCGCTGGCT |
| 841 | CACGCATTGT ATCGGACAGG AGAATATAAG GTCCCAGACG |
| 881 | AGAACGGTGT TCTGGTATCT GGAGGGCTTG ACAAGITGGA |
| 921 | GGGTATGITT GGGTTTATCG ACCAGGTGGA TAAATTCAAC |
| 961 | AACATTAAAA AAAAGTTGAA TAAGCAACCC GACAGATATG |
| 1001 | TTCTGACAAA TGCCACTTTG CACGGATTTA AGCTCAAATT |
| 1041 | GAACGCCAGG GAGAAAGCCT ATAGCAAATT CATCTACTAC |
| 1081 | AAATTCTTCC ACGGTAATAC TTGTCCCACG ATCATAACAG |
| 1121 | AGGGTAAGAC GGATAGGATT TACCTTAAAG CTGCCCTCCA |
| 1161 | TAGCCTCGAG ACAAGTTATC CTGAACTGTT TCGGGAGAAA |
| 1201 | ACAGATAGTA AGAAGAAGGA GATAAATCTG AATATTTTTA |
| 1241 | AAAGCAATGA GAAGACCAAG TATTTCCTGG ATCTCAGCGG |
| 1281 | CGGCACAGCA GACCTCAAGA AATTCGTGGA ACGCTACAAA |
| 1321 | AATAACTACG CTTCCTATTA CGGCAGCGTA CCGAAACAAC |
| 1361 | CGGTGATAAT GGTGCTTGAT AACGACACAG GCCCGTCAGA |
| 1401 | CCTGTTGAAC TTTTTGAGAA ACAAAGTTAA GAGTTGTCCA |
| 1441 | GATGATGTAA CAGAAATGCG CAAGATGAAG TACATACATG |
| 1481 | TGTTTTACAA TCTGTACATA GTTCTGACTC CCCTGTCTCC |
| 1521 | ATCTGGAGAG CAAACGTCTA TGGAGGACCT CTTTCCTAAA |
| 1561 | GATATATTGG ACATTAAGAT AGATGGCAAG AAATTCAATA |
| 1601 | AAAACAATGA CGGTGACTCC AAAACAGAGT ATGGGAAGCA |
| 1001 | CATATTCTCA ATGCGCGTTG TACGAGATAA AAAGAGGAAG |
| 1001 | ATAGATTTCA AGGCATTTTG CTGTATCTTC GATGCTATTA |
| 1001 | AGGATATTAA AGAACATTAC AAACTGATGT TGAATTCCTA |
| 1001 | G |

An example of an Eco1 wild-type retron reverse transcriptase sequence is shown below as SEQ ID NO:4.

| | |
|---|---|
| 1 | KSAEYLNTER LRNLGLPVMN NLHDMSKATR ISVETLRLLI |
| 41 | YTADFRYRIY TVEKKGPEKR MRTIYQPSRE LKALQGWVLR |
| 81 | NILDKLSSSP FSIGFEKHQS ILNNATPHIG ANFILNIDLE |
| 121 | DFFPSLTANK VFGVFHSLGY NRLISSVLTK ICCYKNLLPQ |
| 161 | GAPSSPKLAN LICSKLDYRI QGYAGSRGLI YTRYADDLTL |
| 201 | SAQSMKKVVK ARDFLFSIIP SEGLVINSKK TCISGPRSQR |
| 241 | KVTGLVISQE KVGIGREKYK EIRAKIHHIF CGKSSEIEHV |
| 281 | RGWLSFILSV DSKSHRRLIT YISKLEKKYG KNPLNKAKT |

An example of an Eco2 wild-type retron reverse transcriptase sequence is shown below as SEQ ID NO:5.

| | |
|---|---|
| 1 | MTKTSKLDAL RAATSREDLA KILDIKLVFL TNVLYRIGSD |
| 41 | NQYTQFTIPK KGKGVRTISA PTDRLKDIQR RICDLLSDCR |
| 81 | DEIFAIRKIS NNYSFGFERG KSIILNAYKH RGKQIILNID |
| 121 | LKDFFESFNF GRVRGYFLSN QDFLLNPVVA TTLAKAACYN |
| 161 | GTLPQGSPCS PIISNLICNI MDMRLAKLAK KYGCTYSRYA |
| 201 | DDITISTNKN TFPLEMATVQ PEGVVLGKVL VKEIENSGFE |
| 241 | INDSKTRLTY KTSRQEVTGL TVNRIVNIDR CYYKKTRALA |
| 281 | HALYRTGEYK VPDENGVLVS GGLDKLEGMF GFIDQVDKFN |
| 321 | NIKKKLNKQP DRYVLTNATL HGFKLKLNAR EKAYSKFIYY |
| 361 | KFFHGNTCPT IITEGKTDRI YLKAALHSLE TSYPELFREK |
| 401 | TDSKKKEINL NIFKSNEKTK YFLDLSGGTA DLKKFVERYK |
| 441 | NNYASYYGSV PKQPVIMVLD NDTGPSDLLN FLRNKVKSCP |
| 481 | DDVTEMRKMK YIHVFYNLYI VLTPLSPSGE QTSMEDLFPK |
| 521 | DILDIKIDGK KFNKNNDGDS KTEYGKHIFS MRVVRDKKRK |
| 561 | IDFKAFCCIF DAIKDIKEHY KLMLNS |

An example of a sequence for an Eco4 retron reverse transcriptase is shown below as SEQ ID NO:6.

| | |
|---|---|
| 1 | MSIDIETTLQ KAYPDFDVLL KSRPATHYKV YKIPKRTIGY |
| 41 | RIIAQPTPRV KAIQRDIIEI LKQHTHIHDA ATAYVDGKNI |
| 81 | LDNAKIHQSS VYLLKLDLVN FFNKITPELL FKALARQKVD |
| 121 | ISDTNKNLLK QFCFWNRTKR KNGALVLSVG APSSPFISNI |
| 161 | VMSSFDEEIS SFCKENKISY SRYADDLTFS TNERDVLGLA |
| 201 | HQKVKTTLIR FFGTRIIINN NKIVYSSKAH NRHVTGVTLT |
| 241 | NNNKLSLGRE RKRYITSLVF KFKEGKLSNV DINHLRGLIG |
| 281 | FAYNIEPAFI ERLEKKYGES TIKSIKKYSE GG |

An example of a sequence for a Sen2 retron reverse transcriptase is shown below as SEQ ID NO:7.

| | |
|---|---|
| 1 | MDILQHISDL LLTKKSEIIS FSLTAPYRYK IYKIAKRNSD |
| 41 | KKRTIAHPSK ELKFIQREIT EYLTDKLPVH ECAFAYKKGS |
| 81 | SIKTNAQVHL HTKYLLKMDF ENFFPSITPR LFFSKLRLAN |
| 121 | IDLTADDKVL LENILFFKSK RNSNLRLSIG APSSPLISNF |
| 161 | VMYFWDIEVQ EICSKIGVNY TRYADDLTFS TNNKDVLFDI |
| 201 | PDMLENVLPK YSLGRIRINH EKTVFSSKGH NRHVTGITLT |
| 241 | NDNKLSIGRE RKRKISAMIH HFINGKLSTD ECNKLVGLLA |
| 281 | FAKNIEPSFY KSMVIKYGSD NIYKLQKQKD K |

Other types of retrons are described throughout the application and can be used in the methods described herein.

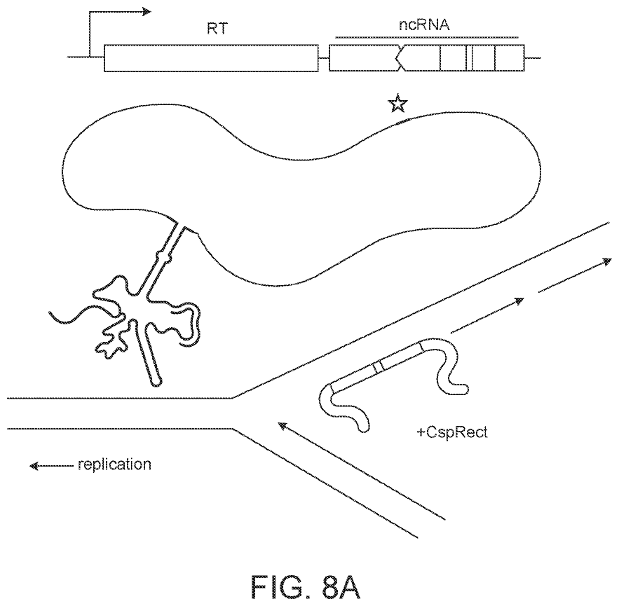

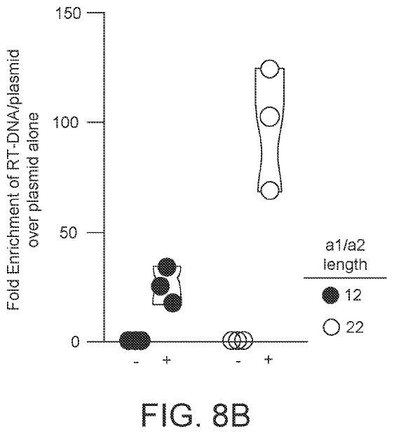

Modified (e.g., engineered) retrons can have alterations in different locations relative to the corresponding wild type retrons. However, not every modification provides a retron that can yield good amounts of reverse transcribed DNA. In some cases, the location of a modification within the retron nucleic acids can affect whether good yields of RT-DNA are obtained. One example of a locus that can often be modified in retron nucleic acids is the loop portion of a stem-loop (see, e.g., FIG. 8A). Another example of a location for modification of retron nucleic acids is within the self-complementary region (stem region, which has sequence complementarity to the pre-msr sequence), wherein the length of the self-complementary region can be lengthened relative to the corresponding region of a native retron. Such modifications can result in engineered retrons that exhibit enhanced production of msDNA.

In certain embodiments, the complementary region has a length at least 1, at least 2, at least 4, at least 6, at least 8, at least 10, at least 12, at least 14, at least 16, at least 18, at least 20, at least 30, at least 40, or at least 50 nucleotides longer than the wild-type self-complementary region. For example, the self-complementary region may have a length ranging from at least 1 to at least 50 nucleotides longer than the native or wild-type complementary region, including any length within this range, such as 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33 34, 35, 36, 37, 38, 39, 40, 41, 42, 43, 44, 45, 46, 47, 48, 49, 50, or more nucleotides longer. In certain embodiments, the self-complementary region has a length ranging from 1 to 16 nucleotides longer than the wild-type complementary region. Such modifications should retain the complementarity of the stem structure.

To create more abundant ssDNA, for example, the ncRNA SEQ ID NO:8 sequence shown below, with the native self-complementary 3′ and 5′ ends highlighted in bold (at positions 1-12 and 158-169), can be extended at positions 1 and 169 to extend the self-complementary region.

| | |
|---|---|
| 1 | TGCGCACCCT TAGCGAGAGG TTTATCATTA AGGTCAACCT |
| 41 | CTGGATGTTG TTTCGGCATC CTGCATTGAA TCTGAGTTAC |
| 81 | TGTCTGTTTT CCTTGTTGGA ACGGAGAGCA TCGCCTGATG |
| 121 | CTCTCCGAGC CAACCAGGAA ACCCGTTTTT TCTGACGTAA |
| 161 | GGGTGCGCA |

For example, as shown below for the following engineered “ncRNA extended” (SEQ ID NO:9), where the additional nucleotides that extend the self-complementary region are shown in italics with underlining.

| | |
|---|---|
| 1 | TGATAAGATT CCGTATGCGC ACCCTTAGCG AGAGGTTTAT |
| 41 | CATTAAGGTC AACCTCTGGA TGTTGTTTCG GCATCCTGCA |
| 81 | TTGAATCTGA GTTACTGTCT GTTTTCCTTG TTGGAACGGA |
| 121 | GAGCATCGCC TGATGCTCTC CGAGCCAACC AGGAAACCCG |
| 161 | TTTTTTCTGA CGTAAGGGTG CGCATACGGA ATCTTATCA |

In some cases, the additional nucleotides can be added to any position in the self-complementary region, for example, anywhere within positions 1-12 and 158-169 of the SEQ ID NO:8 or SEQ ID NO:9 sequence.

In certain embodiments, sequences of the retron nucleic acids (e.g., ncRNA and reverse transcriptase sequences) used in the engineered retron may be derived from any bacterial retron operon. Representative retrons are available such as those from gram-negative bacteria including, without limitation, myxobacteria retrons such as Myxococcus xanthus retrons (e.g., Mx65, Mx162) and Stigmatella aurantiaca retrons (e.g., Sa163); Escherichia coli retrons (e.g., Ec48, E67, Ec73, Ec78, EC83, EC86, EC107, and Ec107); Salmonella enterica; Vibrio cholerae retrons (e.g., Vc81, Vc95, Vc137); Vibrio parahaemolyticus (e.g., Vc96); and Nannocystis exedens retrons (e.g., Ne144). Retron msr gene, msd gene, and ret gene nucleic acid sequences as well as retron reverse transcriptase protein sequences may be derived from any source. Representative retron sequences, including msr gene, msd gene, and ret gene nucleic acid sequences and reverse transcriptase protein sequences are listed in the National Center for Biotechnology Information (NCBI) database. See, for example, NCBI entries: Accession Nos. EF428983, M55249, EU250030, X60206, X62583, AB299445, AB436696, AB436695, M86352, M30609, M24392, AF427793, AQ3354, and AB079134; all of which sequences (as entered by the date of filing of this application) are herein incorporated by reference in their entireties.

The retron ncRNAs can be modified to enhance production of retron reverse transcribed DNA in a host cell or to provide host cells with genomic editing components or other useful proteins and/or nucleic acids. Any of the foregoing retron sequences (or variants thereof) can include variant or mutant nucleotides, added nucleotides, or fewer nucleotides.

For example, a parental ncRNA can be modified by addition of nucleotides to a stem or loop as described herein. Before modification the parental ncRNA can have at least about 80-100% sequence identity to any region of the retrons described herein, including any percent identity within this range, such as 81, 82, 83, 84, 85, 86, 87, 88, 89, 90, 91, 92, 93, 94, 95, 96, 97, 98, or 99% sequence identity to any region of the retron sequences described herein (including those defined by accession number). Such parental retrons can be used to construct an engineered retron or vector system comprising an engineered retron, as described herein.

The variant retrons can include exogenous or heterologous nucleotide or nucleic acid segments. For example, the exogenous or heterologous nucleotide or nucleic acid segments can add at least 1, at least 2, at least 4, at least 6, at least 8, at least 10, at least 12, at least 14, at least 16, at least 18, at least 20, at least 30, at least 40, at least 50, at least 60, at least 70, at least 80, at least 90, at least 100, at least 125, at least 150, at least 175, or at least 200 nucleotides to parental retron nucleic acids, to thereby generate variant retron nucleic acids.

In some embodiments, recombinant retron constructs have a non-native configuration with a non-native spacing between the msr gene, msd gene, and ret gene. The msr gene and the msd gene may be separated in a trans arrangement rather than provided in the endogenous cis arrangement. For example, in many cases the ret gene (reverse transcriptase) may be provided in a trans arrangement with respect to either the ncRNA, msr gene or the msd gene. In some embodiments, the ret gene is provided in a trans arrangement so that it is expressed from a different expression cassette or expression vector from the ncRNA. For example, use of a ret gene expressed in trans arrangement from the ncRNA eliminates a cryptic stop signal for the reverse transcriptase, which allows the generation of longer single stranded DNAs from the engineered ncRNA retron construct.

As described herein, the retron nucleic acids can be modified with respect to the native retron to include a heterologous sequence of interest. The retrons can be engineered with heterologous sequences for use in a variety of applications. For example, heterologous sequences can be added to retron constructs to provide a cell with a nucleic acid encoding a protein or regulatory RNA of interest, a donor polynucleotide suitable for use in gene editing (e.g., by homology directed repair (HDR) or recombination-mediated genetic engineering, recombineering), a guide RNA, or a CRISPR protospacer DNA sequence for use in molecular recording, or a combination thereof. Such heterologous sequences may be inserted, for example, into the msr gene or the msd gene such that the heterologous sequence is transcribed by the retron reverse transcriptase as part of the msDNA product. In some cases, the heterologous sequence of interest can be inserted into the loop of the msd stem loop.

In some cases, engineered retron nucleic acids can include unique barcodes to facilitate multiplexing. For example, in some cases barcodes can be used to identify particular retrons, retron variants, or retron modifications. Barcodes may comprise one or more nucleotide sequences that are used to identify a nucleic acid or cell with which the barcode is associated. Such barcodes may be inserted for example, into the loop region of the ncRNA. Barcodes can be 3-1000 or more nucleotides in length, preferably 10-250 nucleotides in length, and more preferably 10-30 nucleotides in length, including any length within these ranges, such as 3, 4, 5, 6, 7, 8, 9, 10, 11, 12, 13, 14, 15, 16, 17, 18, 19, 20, 21, 22, 23, 24, 25, 26, 27, 28, 29, 30, 40, 50, 60, 70, 80, 90, 100, 200, 300, 400, 500, 600, 700, 800, 900, or 1000 nucleotides in length. In some embodiments, barcodes can be used to identify the position (i.e., positional barcode) of a cell, colony, or sample from which a retron originated, such as the position of a colony in a cellular array, the position of a well in a multi-well plate, the position of a tube in a rack, or the location of a sample in a laboratory. In particular, a barcode may be used to identify the identity or position of a genetically modified cell containing a retron. The use of barcodes allows retrons from different cells to be pooled in a single reaction mixture for sequencing while still being able to trace a particular retron back to the colony from which it originated.

Amplification of retron nucleic acids may be performed, for example, before introduction into cells or ligation into vectors. Any method for amplifying the retron constructs may be used, including, but not limited to polymerase chain reaction (PCR), isothermal amplification, nucleic acid sequence-based amplification (NASBA), transcription mediated amplification (TMA), strand displacement amplification (SDA), and ligase chain reaction (LCR). In one embodiment, the retron constructs comprise common 5′ and 3′ priming sites to allow amplification of retron sequences in parallel with a set of universal primers. In another embodiment, a set of selective primers is used to selectively amplify a subset of retron sequences from a pooled mixture.

### Libraries

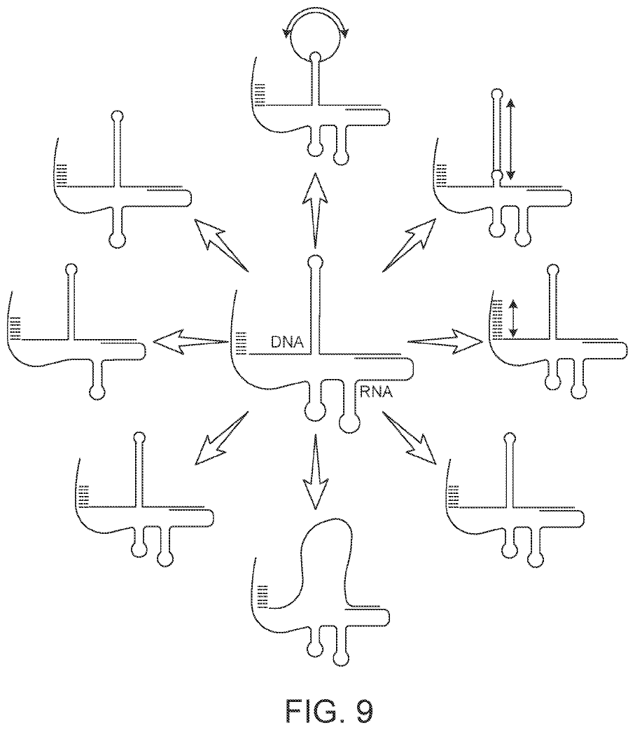

Libraries of retrons or retron nucleic acids can be used in the methods described herein. Tens of thousands of retron variants or mutants were synthesized to systematically test each structural variant (see, e.g., FIG. 9). A golden-gate-based cloning strategy (Engler et al., PLOS One (Nov. 5, 2008)) can be used to clone these variants, and then large pools of modified retrons can be expressed in a multiplexed vectors along with the reverse transcriptase.

For example, a plasmid having or encoding a parental retron nucleic acid insert (e.g., having a parental RT-DNA or encoding a parental ncRNA) can be subjected to mutagenesis to generate a population of plasmids with different mutant or variant retron nucleic acids. The plasmid can be an expression vector or the different mutant or variant retron nucleic acid inserts can be inserted into expression vectors or expression cassettes so that the variant retron nucleic acid inserts can be expressed to generate the retron nucleic acids (ncRNAs, RT-DNAs) that are evaluated using the methods described herein.

Alternatively, a population of retron nucleic acid oligonucleotides can be subjected to mutagenesis to generate a population of retron variant nucleic acid oligonucleotides, which can be inserted into expression vectors or expression cassettes so that the variant retron nucleic acid inserts can be expressed to generate the retron nucleic acids that are evaluated using the methods described herein. In some cases, the retron variant nucleic acid oligonucleotides can be a population of different retron nucleic acids that are evaluated using the methods described herein.

### Retron Expression Systems

Retrons or retron nucleic acids can be incorporated into and expressed from an expression cassette or expression vector. In general, the selected retron nucleic acids are one or more wild type, mutant, or variant ncRNA, or populations thereof. The retrons or retron libraries can be expressed from expression cassettes or expression vectors that can be within host cells.

The retron ncRNAs, msr gene, msd gene, and/or ret gene can individually or collectively be expressed in vivo from a vector within a cell. A “vector” is a composition of matter which can be used to deliver a nucleic acid of interest to the interior of a cell. The retron nucleic acids can be introduced into a cell with a single vector or in multiple separate vectors to produce RT-DNA in host cells. Vectors typically include control elements operably linked to the retron sequences, which allow for the production of ncRNAs and hence RT-DNA in vivo in the host cells. For example, the segment encoding the retron ncRNA and/or the segment encoding the ret (reverse transcriptase) can be operably linked to a promoter to allow expression of the ncRNA and/or the retron reverse transcriptase. When both the ncRNA and the retron reverse transcriptase are expressed in a host cell, retron RT-DNA can be produced. The types and amounts of RT-DNA so produced can be analyzed to determine which variants and combinations of retron sequences are desirable.

In some embodiments, heterologous sequences encoding desired products of interest (e.g., polynucleotide encoding polypeptide or regulatory RNA, donor polynucleotide for gene editing, guide RNA, or protospacer DNA for molecular recording) may be inserted in the segment encoding the ncRNA.

Any eukaryotic, archeon, or prokaryotic cell, capable of being transfected with a vector comprising the engineered retron sequences, may be used to produce the RT-DNA. The ability of constructs to produce the RT-DNA along with other retron-encoded products can be empirically determined using the methods described herein.

In some embodiments, the engineered retron is produced by a vector system comprising one or more vectors. In the vector system, the ncRNA and the reverse transcriptase may be provided by the same vector (i.e., cis arrangement of such retron elements), wherein the vector comprises a promoter operably linked to the segment encoding the ncRNA and the segment encoding the reverse transcriptase. In some embodiments, a second promoter is operably linked to the segment encoding the reverse transcriptase. Alternatively, the segment encoding the reverse transcriptase may be incorporated into a second vector that does not include the ncRNA, msr gene and the msd gene (i.e., trans arrangement).

Numerous vectors can be used including, but not limited to, linear polynucleotides, polynucleotides associated with ionic or amphiphilic compounds, plasmids, and viruses. Thus, the term “vector” includes an autonomously replicating plasmid or a virus. Examples of viral vectors include, but are not limited to, adenoviral vectors, adeno-associated virus vectors, retroviral vectors, lentiviral vectors, and the like. An expression construct can be replicated in a living cell, or it can be made synthetically. For purposes of this application, the terms “expression construct,” “expression vector,” and “vector,” are used interchangeably to demonstrate the application of the invention in a general, illustrative sense, and are not intended to limit the invention.

The nucleic acid comprising one or more retron sequences can be under transcriptional control of a promoter. A “promoter” refers to a DNA sequence recognized by the synthetic machinery of the cell, or introduced synthetic machinery, required to initiate the specific transcription of a gene. The term promoter will be used here to refer to a group of transcriptional control modules that are clustered around the initiation site for RNA polymerase I, II, or III. Typical promoters for mammalian cell expression include the SV40 early promoter, a CMV promoter such as the CMV immediate early promoter (see, U.S. Pat. Nos. 5,168,062 and 5,385,839, incorporated herein by reference in their entireties), the mouse mammary tumor virus LTR promoter, the adenovirus major late promoter (Ad MLP), and the herpes simplex virus promoter, among others. Other nonviral promoters, such as a promoter derived from the murine metallothionein gene, will also find use for mammalian expression. These and other promoters can be obtained from commercially available plasmids, using techniques well known in the art. See, e.g., Sambrook et al., supra. Enhancer elements may be used in association with the promoter to increase expression levels of the constructs. Examples include the SV40 early gene enhancer, as described in Dijkema et al., EMBO J. (1985) 4:761, the enhancer/promoter derived from the long terminal repeat (LTR) of the Rous Sarcoma Virus, as described in Gorman et al., Proc. Natl. Acad Sci. USA (1982b) 79:6777 and elements derived from human CMV, as described in Boshart et al., Cell (1985) 41:521, such as elements included in the CMV intron A sequence.

Expression vectors for expressing one or more retron nucleic acids can include a promoter “operably linked” to a nucleic acid segment encoding the ncRNA and/or the reverse transcriptase. The phrase “operably linked” or “under transcriptional control” as used herein means that the promoter is in the correct location and orientation in relation to a polynucleotide to control the initiation of transcription by RNA polymerase and expression of the ncRNA and/or the reverse transcriptase.

Typically, transcription terminator/polyadenylation signals will also be present in the expression construct. Examples of such sequences include, but are not limited to, those derived from SV40, as described in Sambrook et al., supra, as well as a bovine growth hormone terminator sequence (see, e.g., U.S. Pat. No. 5,122,458). Additionally, 5′-UTR sequences can be placed adjacent to the coding sequence in order to enhance expression of the same. Such sequences may include UTRs comprising an internal ribosome entry site (IRES).

Inclusion of an IRES permits the translation of one or more open reading frames from a vector. Such an IRES element attracts a eukaryotic ribosomal translation initiation complex and promotes translation initiation. See, e.g., Kaufman et al., Nuc. Acids Res. (1991) 19:4485-4490; Gurtu et al., Biochem. Biophys. Res. Comm. (1996) 229:295-298; Rees et al., BioTechniques (1996) 20:102-110; Kobayashi et al., BioTechniques (1996) 21:399-402; and Mosser et al., BioTechniques (1997 22: 150-161. A multitude of IRES sequences are available and include sequences derived from a wide variety of viruses, such as from leader sequences of picornaviruses such as the encephalomyocarditis virus (EMCV) UTR (Jang et al. J Virol. (1989) 63:1651-1660), the polio leader sequence, the hepatitis A virus leader, the hepatitis C virus IRES, human rhinovirus type 2 IRES (Dobrikova et al., Proc. Natl. Acad. Sci. (2003) 100(25):15125-15130), an IRES element from the foot and mouth disease virus (Ramesh et al., Nucl. Acid Res. (1996) 24:2697-2700), a giardiavirus IRES (Garlapati et al., J Biol. Chem. (2004) 279(5):3389-3397), and the like. A variety of nonviral IRES sequences will also find use herein, including, but not limited to IRES sequences from yeast, as well as the human angiotensin II type 1 receptor IRES (Martin et al., Mol. Cell Endocrinol. (2003) 212:51-61), fibroblast growth factor IRESs (FGF-1 IRES and FGF-2 IRES, Martineau et al. (2004) Mol. Cell. Biol. 24(17):7622-7635), vascular endothelial growth factor IRES (Baranick et al. (2008) Proc. Natl. Acad Sci. U.S.A. 105(12):4733-4738, Stein et al. (1998) Mol. Cell. Biol. 18(6):3112-3119, Bert et al. (2006) RNA 12(6):1074-1083), and insulin-like growth factor 2 IRES (Pedersen et al. (2002) Biochem. J. 363 (Pt 1):37-44). These elements are readily commercially available in plasmids sold, e.g., by Clontech (Mountain View, CA), Invivogen (San Diego, CA), Addgene (Cambridge, MA) and GeneCopoeia (Rockville, MD). See also IRESite: The database of experimentally verified IRES structures (iresite.org). An IRES sequence may be included in a vector, for example, to express a reverse transcriptase or an RNA-guided nuclease (e.g., Cas9) from an expression cassette.

Alternatively, a polynucleotide encoding a viral 2A cleavable peptide can be used to allow production of multiple protein products (e.g., Cas9, bacteriophage recombination proteins, retron reverse transcriptase) from a single vector. One or more 2A linker peptides can be inserted between the coding sequences in the multicistronic construct. The 2A peptide, which is self-cleaving, allows co-expressed proteins from the multicistronic construct to be produced at equimolar levels. 2A peptides from various viruses may be used, including, but not limited to 2A peptides derived from the foot-and-mouth disease virus, equine rhinitis A virus, Thosea asigna virus and porcine teschovirus-1. See, e.g., Kim et al. (2011) PLoS One 6(4):e18556, Trichas et al. (2008) BMC Biol. 6:40, Provost et al. (2007) Genesis 45(10):625-629, Furler et al. (2001) Gene Ther. 8(11):864-873; herein incorporated by reference in their entireties.

In certain embodiments, the expression construct comprises a plasmid sequences suitable for transforming a bacterial host. Numerous bacterial expression vectors are available. Bacterial expression vectors include, but are not limited to, pACYC177, pASK75, pBAD, pBADM, pBAT, pCal, pET, pETM, pGAT, pGEX, pHAT, pKK223, pMal, pProEx, pQE, and pZA31. Bacterial plasmids may contain antibiotic selection markers (e.g., ampicillin, kanamycin, erythromycin, carbenicillin, streptomycin, or tetracycline resistance), a lacZ gene (β-galactosidase produces blue pigment from x-gal substrate), fluorescent markers (e.g., GFP. mCherry), or other markers for selection of transformed bacteria. See, e.g., Sambrook et al., supra.

In other embodiments, the expression construct comprises a plasmid suitable for transforming a yeast cell. Yeast expression plasmids typically contain a yeast-specific origin of replication (ORI) and nutritional selection markers (e.g., HIS3, URA3, LYS2, LEU2, TRP1, MET15, ura4+, leu1+, ade6+), antibiotic selection markers (e.g., kanamycin resistance), fluorescent markers (e.g., mCherry), or other markers for selection of transformed yeast cells. The yeast plasmid may further contain components to allow shuttling between a bacterial host (e.g., E. coli) and yeast cells. A number of different types of yeast plasmids are available including yeast integrating plasmids (YIp), which lack an ORI and are integrated into host chromosomes by homologous recombination; yeast replicating plasmids (YRp), which contain an autonomously replicating sequence (ARS) and can replicate independently; yeast centromere plasmids (YCp), which are low copy vectors containing a part of an ARS and part of a centromere sequence (CEN); and yeast episomal plasmids (YEp), which are high copy number plasmids comprising a fragment from a 2 micron circle (a natural yeast plasmid) that allows for 50 or more copies to be stably propagated per cell.

In other embodiments, the expression construct comprises a virus or engineered construct derived from a viral genome. A number of viral based systems have been developed for gene transfer into mammalian cells. These include adenoviruses, retroviruses (γ-retroviruses and lentiviruses), poxviruses, adeno-associated viruses, baculoviruses, and herpes simplex viruses (see e.g., Warnock et al. (2011) Methods Mol. Biol. 737:1-25; Walther et al. (2000) Drugs 60(2):249-271; and Lundstrom (2003) Trends Biotechnol. 21(3):117-122; herein incorporated by reference in their entireties). The ability of certain viruses to enter cells via receptor-mediated endocytosis, to integrate into host cell genomes and express viral genes stably and efficiently have made them attractive candidates for the transfer of foreign genes into mammalian cells.

For example, retroviruses provide a convenient platform for gene delivery systems. Selected sequences can be inserted into a vector and packaged in retroviral particles. The recombinant virus can then be isolated and delivered to host cells, or cells of a selected subject either in vivo or ex vivo. A number of retroviral systems have been described (U.S. Pat. No. 5,219,740; Miller and Rosman (1989) BioTechniques 7:980-990; Miller, A. D. (1990) Human Gene Therapy 1:5-14; Scarpa et al. (1991) Virology 180:849-852; Burns et al. (1993) Proc. Natl. Acad. Sci. USA 90:8033-8037; Boris-Lawrie and Temin (1993) Cur. Opin. Genet. Develop. 3:102-109; and Ferry et al. (2011) Curr. Pharm. Des. 17(24):2516-2527). Lentiviruses are a class of retroviruses that are particularly useful for delivering polynucleotides to mammalian cells because they are able to infect both dividing and nondividing cells (see e.g., Lois et a1 (2002) Science 295:868-872; Durand et al. (2011) Viruses 3(2):132-159; herein incorporated by reference).

A number of adenovirus vectors have also been described. Unlike retroviruses which integrate into the host genome, adenoviruses persist extrachromosomally thus minimizing the risks associated with insertional mutagenesis (Haj-Ahmad and Graham, J. Virol. (1986) 57:267-274; Bett et al., J. Virol. (1993) 67:5911-5921; Mittereder et al., Human Gene Therapy (1994) 5:717-729; Seth et al., J. Virol. (1994) 68:933-940; Barr et al., Gene Therapy (1994) 1:51-58; Berkner, K. L. BioTechniques (1988) 6:616-629; and Rich et al., Human Gene Therapy (1993) 4:461-476). Additionally, various adeno-associated virus (AAV) vector systems have been developed for gene delivery. AAV vectors can be readily constructed using techniques well known in the art. See, e.g., U.S. Pat. Nos. 5,173,414 and 5,139,941; International Publication Nos. WO 92/01070 (published 23 Jan. 1992) and WO 93/03769 (published 4 Mar. 1993); Lebkowski et al., Molec. Cell. Biol. (1988) 8:3988-3996; Vincent et al., Vaccines 90 (1990) (Cold Spring Harbor Laboratory Press); Carter, B. J. Current Opinion in Biotechnology (1992) 3:533-539; Muzyczka, N. Current Topics in Microbiol. and Immunol. (1992) 158:97-129; Kotin, R. M. Human Gene Therapy (1994) 5:793-801; Shelling and Smith, Gene Therapy (1994) 1:165-169; and Zhou et al., J. Exp. Med. (1994) 179:1867-1875.

Another vector system useful for delivering nucleic acids encoding the engineered retrons is the enterically administered recombinant poxvirus vaccines described by Small, Jr., P. A., et al. (U.S. Pat. No. 5,676,950, issued Oct. 14, 1997, herein incorporated by reference).

Additional viral vectors which will find use for delivering the nucleic acid molecules of interest include those derived from the pox family of viruses, including vaccinia virus and avian poxvirus. By way of example, vaccinia virus recombinants expressing a nucleic acid molecule of interest (e.g., engineered retron) can be constructed as follows. The DNA encoding the particular nucleic acid sequence is first inserted into an appropriate vector so that it is adjacent to a vaccinia promoter and flanking vaccinia DNA sequences, such as the sequence encoding thymidine kinase (TK). This vector is then used to transfect cells which are simultaneously infected with vaccinia. Homologous recombination serves to insert the vaccinia promoter plus the gene encoding the sequences of interest into the viral genome. The resulting TK-recombinant can be selected by culturing the cells in the presence of 5-bromodeoxyuridine and picking viral plaques resistant thereto.

Alternatively, avipoxviruses, such as the fowlpox and canarypox viruses, can also be used to deliver the nucleic acid molecules of interest. The use of an avipox vector is particularly desirable in human and other mammalian species since members of the avipox genus can only productively replicate in susceptible avian species and therefore are not infective in mammalian cells. Methods for producing recombinant avipoxviruses are known in the art and employ genetic recombination, as described above with respect to the production of vaccinia viruses. See, e.g., WO 91/12882; WO 89/03429; and WO 92/03545.

Molecular conjugate vectors, such as the adenovirus chimeric vectors described in Michael et al., J. Biol. Chem. (1993) 268:6866-6869 and Wagner et al., Proc. Natl. Acad. Sci. USA (1992) 89:6099-6103, can also be used for gene delivery.

Members of the alphavirus genus, such as, but not limited to, vectors derived from the Sindbis virus (SIN), Semliki Forest virus (SFV), and Venezuelan Equine Encephalitis virus (VEE), will also find use as viral vectors for delivering the polynucleotides of the present invention. For a description of Sindbis-virus derived vectors useful for the practice of the instant methods, see, Dubensky et al. (1996) J. Virol. 70:508-519; and International Publication Nos. WO 95/07995, WO 96/17072; as well as Dubensky, Jr., T. W., et al., U.S. Pat. No. 5,843,723, issued Dec. 1, 1998, and Dubensky, Jr., T. W., U.S. Pat. No. 5,789,245, issued Aug. 4, 1998, both herein incorporated by reference. Particularly preferred are chimeric alphavirus vectors comprised of sequences derived from Sindbis virus and Venezuelan equine encephalitis virus. See, e.g., Perri et al. (2003) J. Virol. 77: 10394-10403 and International Publication Nos. WO 02/099035, WO 02/080982, WO 01/81609, and WO 00/61772; herein incorporated by reference in their entireties.

A vaccinia-based infection/transfection system can be conveniently used to provide for inducible, transient expression of the nucleic acids of interest (e.g., engineered retron) in a host cell. In this system, cells are first infected in vitro with a vaccinia virus recombinant that encodes the bacteriophage T7 RNA polymerase. This polymerase displays exquisite specificity in that it only transcribes templates bearing T7 promoters. Following infection, cells are transfected with the nucleic acid of interest, driven by a T7 promoter. The polymerase expressed in the cytoplasm from the vaccinia virus recombinant transcribes the transfected DNA into RNA. The method provides for high level, transient, cytoplasmic production of large quantities of RNA. See, e.g., Elroy-Stein and Moss, Proc. Natl. Acad. Sci. USA (1990) 87:6743-6747; Fuerst et al., Proc. Natl. Acad. Sci. USA (1986) 83:8122-8126.

As an alternative approach to infection with vaccinia or avipox virus recombinants, or to the delivery of nucleic acids using other viral vectors, an amplification system can be used that will lead to high level expression following introduction into host cells. Specifically, a T7 RNA polymerase promoter preceding the coding region for T7 RNA polymerase can be engineered. Translation of RNA derived from this template will generate T7 RNA polymerase which in turn will transcribe more templates. Concomitantly, there will be a DNA encoding the T7 RNA polymerase (and DNAs encoding the ncRNAs, reverse transcriptases) whose expression is under the control of the T7 promoter. Thus, some of the T7 RNA polymerase generated from translation of the amplification template RNA will lead to transcription of the desired gene. Because some T7 RNA polymerase is required to initiate the amplification, T7 RNA polymerase can be introduced into cells along with the template(s) to prime the transcription reaction. The polymerase can be introduced as a protein or on a plasmid encoding the RNA polymerase. For a further discussion of T7 systems and their use for transforming cells, see, e.g., International Publication No. WO 94/26911; Studier and Moffatt, J. Mol. Biol. (1986) 189:113-130; Deng and Wolff, Gene (1994) 143:245-249; Gao et al., Biochem. Biophys. Res. Commun. (1994) 200:1201-1206; Gao and Huang, Nuc. Acids Res. (1993) 21:2867-2872; Chen et al., Nuc. Acids Res. (1994) 22:2114-2120; and U.S. Pat. No. 5,135,855.

Insect cell expression systems, such as baculovirus systems, can also be used and are known to those of skill in the art and described in, e.g., Baculovirus and Insect Cell Expression Protocols (Methods in Molecular Biology, D. W. Murhammer ed., Humana Press, 2^{nd} edition, 2007) and L. King, The Baculovirus Expression System: A laboratory guide (Springer, 1992). Materials and methods for baculovirus/insect cell expression systems are commercially available in kit form from, inter alia, Thermo Fisher Scientific (Waltham, MA) and Clontech (Mountain View, CA).

Plant expression systems can also be used for transforming plant cells. Generally, such systems use virus-based vectors to transfect plant cells with heterologous genes. For a description of such systems see, e.g., Porta et al., Mol. Biotech. (1996) 5:209-221; and Hackland et al., Arch. Virol. (1994) 139:1-22.

In order to effect expression of engineered retron constructs, the expression construct can be delivered into a cell. This delivery may be accomplished in vitro, as in laboratory procedures for transforming cells lines.

One mechanism for delivery is via viral infection where the expression construct is encapsulated in an infectious viral particle. Several non-viral methods for the transfer of expression constructs into cultured cells also are contemplated. These include the use of calcium phosphate precipitation, DEAE-dextran, electroporation, direct microinjection, DNA-loaded liposomes, lipofectamine-DNA complexes, cell sonication, gene bombardment using high velocity microprojectiles, and receptor-mediated transfection (see, e.g., Graham and Van Der Eb (1973) Virology 52:456-467; Chen and Okayama (1987) Mol. Cell Biol. 7:2745-2752; Rippe et al. (1990) Mol. Cell Biol. 10:689-695; Gopal (1985) Mol. Cell Biol. 5:1188-1190; Tur-Kaspa et al. (1986) Mol. Cell. Biol. 6:716-718; Potter et al. (1984) Proc. Natl. Acad. Sci. USA 81:7161-7165); Harland and Weintraub (1985) J. Cell Biol. 101:1094-1099); Nicolau & Sene (1982) Biochim. Biophys. Acta 721:185-190; Fraley et al. (1979) Proc. Natl. Acad. Sci. USA 76:3348-3352; Fechheimer et al. (1987) Proc Natl. Acad. Sci. USA 84:8463-8467; Yang et al. (1990) Proc. Natl. Acad. Sci. USA 87:9568-9572; Wu and Wu (1987) J. Biol. Chem. 262:4429-4432; Wu and Wu (1988) Biochemistry 27:887-892; herein incorporated by reference).

Delivery of retron nucleic acids to a cell can generally be accomplished with or without vectors. The retrons, retron nucleic acids, or vectors containing them may be introduced into any type of cell, including any cell from a prokaryotic, eukaryotic, or archaeon organism, including bacteria, archaea, fungi, protists, plants (e.g., monocotyledonous and dicotyledonous plants); and animals (e.g., vertebrates and invertebrates). Examples of animal host cells that may be transfected with an engineered retron include, without limitation, vertebrates such as fish, birds, mammals (e.g., human and non-human primates, farm animals, pets, and laboratory animals), reptiles, and amphibians. Examples of plant cells that may be transfected with an engineered retron include, without limitation, crops including cereals such as wheat, oats, and rice, legumes such as soybeans and peas, corn, grasses such as alfalfa, and cotton. The engineered retrons can be introduced into a single cell or a population of cells of interest. Cells from tissues, organs, and biopsies, as well as recombinant cells, genetically modified cells, cells from cell lines cultured in vitro, and artificial cells (e.g., nanoparticles, liposomes, polymersomes, or microcapsules encapsulating nucleic acids) may all be transfected with the engineered retrons. The subject methods are also applicable to cellular fragments, cell components, or organelles (e.g., mitochondria in animal and plant cells, plastids (e.g., chloroplasts) in plant cells and algae). Cells may be cultured or expanded after transfection with the engineered retron constructs.

A variety of methods for introducing nucleic acids into a host cell are available. Commonly used methods include chemically induced transformation, typically using divalent cations (e.g., CaCl₂)), dextran-mediated transfection, polybrene mediated transfection, lipofectamine and LT-1 mediated transfection, electroporation, protoplast fusion, encapsulation of nucleic acids in liposomes, and direct microinjection of the nucleic acids comprising engineered retrons into nuclei. See, e.g., Sambrook et al. (2001) Molecular Cloning, a laboratory manual, 3^{rd} edition, Cold Spring Harbor Laboratories, New York, Davis et al. (1995) Basic Methods in Molecular Biology, 2^{nd} edition, McGraw-Hill, and Chu et al. (1981) Gene 13:197; herein incorporated by reference in their entireties.

Once the expression constructs have been delivered into the cell the vectors or cassettes comprising the retron nucleic acids may be positioned and expressed at different sites. In certain embodiments, the vector or cassette comprising the retron nucleic acids may be stably integrated into the genome of the cell. This integration may be in the cognate location and orientation, or it may be integrated in a random, non-specific location (gene augmentation). In yet further embodiments, the vector or cassette comprising the retron nucleic acids may be stably maintained in the cell as a separate, episomal segment of DNA. Such nucleic acid segments or “episomes” encode sequences sufficient to permit maintenance and replication independent of or in synchronization with the host cell cycle. How the vector or cassette comprising the retron nucleic acids are delivered to a cell and where in the cell the nucleic acid remains is dependent on the type of expression construct employed.

In yet another embodiment, the expression construct may simply consist of naked recombinant DNA or plasmids comprising the retron nucleic acids. Transfer of the construct may be performed by any of the methods mentioned above which physically or chemically permeabilize the cell membrane. This is particularly applicable for transfer in vitro but it may be applied to in vivo use as well. Dubensky et al. (Proc. Natl. Acad. Sci. USA (1984) 81:7529-7533) successfully injected polyomavirus DNA in the form of calcium phosphate precipitates into liver and spleen of adult and newborn mice demonstrating active viral replication and acute infection. Benvenisty & Neshif (Proc. Natl. Acad. Sci. USA (1986) 83:9551-9555) also demonstrated that direct intraperitoneal injection of calcium phosphate-precipitated plasmids results in expression of the transfected genes. It is envisioned that DNA encoding retron nucleic acids of interest such as ncRNAs and reverse transcriptases may be transferred in a similar manner to co-express the retron products.

In still another embodiment, a naked DNA expression construct may be transferred into cells by particle bombardment. This method depends on the ability to accelerate DNA-coated microprojectiles to a high velocity allowing them to pierce cell membranes and enter cells without killing them (Klein et al. (1987) Nature 327:70-73). Several devices for accelerating small particles have been developed. One such device relies on a high voltage discharge to generate an electrical current, which in turn provides the motive force (Yang et al. (1990) Proc. Natl. Acad. Sci. USA 87:9568-9572). The microprojectiles may consist of biologically inert substances, such as tungsten or gold beads.

In a further embodiment, the expression constructs may be delivered using liposomes. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh & Bachhawat (1991) Liver Diseases, Targeted Diagnosis and Therapy Using Specific Receptors and Ligands, Wu et al. (Eds.), Marcel Dekker, NY, 87-104). Also contemplated is the use of lipofectamine-DNA complexes.

In certain embodiments, the liposomes may be complexed with a hemagglutinating virus (HVJ). This has been shown to facilitate fusion with the cell membrane and promote cell entry of liposome-encapsulated DNA (Kaneda et al. (1989) Science 243:375-378). In other embodiments, the liposomes may be complexed or employed in conjunction with nuclear non-histone chromosomal proteins (HMG-I) (Kato et al. (1991) J. Biol. Chem. 266(6):3361-3364). In yet further embodiments, the liposomes may be complexed or employed in conjunction with both HVJ and HMG-I. In that such expression constructs have been successfully employed in transfer and expression of nucleic acid in vitro and in vivo, then they are applicable for the present invention. Where a bacterial promoter is employed in the DNA construct, may also be desirable to include within the liposome an appropriate bacterial polymerase.

Other expression constructs which can be employed to deliver a nucleic acid into cells are receptor-mediated delivery vehicles. These take advantage of the selective uptake of macromolecules by receptor-mediated endocytosis in almost all eukaryotic cells. Because of the cell type-specific distribution of various receptors, the delivery can be highly specific (Wu and Wu (1993) Adv. Drug Delivery Rev. 12:159-167).

Receptor-mediated gene targeting vehicles generally consist of two components: a cell receptor-specific ligand and a DNA-binding agent. Several ligands have been used for receptor-mediated gene transfer. The most extensively characterized ligands are asialoorosomucoid (ASOR) and transferrin (see, e.g., Wu and Wu (1987), supra; Wagner et al. (1990) Proc. Natl. Acad. Sci. USA 87(9):3410-3414). A synthetic neoglycoprotein, which recognizes the same receptor as ASOR, has been used as a gene delivery vehicle (Ferkol et al. (1993) FASEB J. 7:1081-1091; Perales et al. (1994) Proc. Natl. Acad. Sci. USA 91(9):4086-4090), and epidermal growth factor (EGF) has also been used to deliver genes to squamous carcinoma cells (Myers, EPO 0273085).

In other embodiments, the delivery vehicle may comprise a ligand and a liposome. For example, Nicolau et al. (Methods Enzymol. (1987) 149:157-176) employed lactosyl-ceramide, a galactose-terminal asialoganglioside, incorporated into liposomes and observed an increase in the uptake of the insulin gene by hepatocytes. Thus, it is feasible that a nucleic acid encoding a particular gene also may be specifically delivered into a cell by any number of receptor-ligand systems with or without liposomes. Also, antibodies to surface antigens on cells can similarly be used as targeting moieties.

In a particular example, a recombinant polynucleotide comprising retron nucleic acids may be administered in combination with a cationic lipid. Examples of cationic lipids include, but are not limited to, lipofectin, DOTMA, DOPE, and DOTAP. The publication of WO/0071096, which is specifically incorporated by reference, describes different formulations, such as a DOTAP:cholesterol or cholesterol derivative formulation that can effectively be used for gene therapy. Other disclosures also discuss different lipid or liposomal formulations including nanoparticles and methods of administration; these include, but are not limited to, U.S. Patent Publication 20030203865, 20020150626, 20030032615, and 20040048787, which are specifically incorporated by reference to the extent they disclose formulations and other related aspects of administration and delivery of nucleic acids. Methods used for forming particles are also disclosed in U.S. Pat. Nos. 5,844,107, 5,877,302, 6,008,336, 6,077,835, 5,972,901, 6,200,801, and 5,972,900, which are incorporated by reference for those aspects.

### Definitions

The term “about” as used herein when referring to a measurable value such as an amount, a length, and the like, is meant to encompass variations of +20% or +10%, more preferably +5%, even more preferably +1%, and still more preferably +0.1% from the specified value.

“Recombinant” as used herein to describe a nucleic acid molecule means a polynucleotide of retron, genomic, cDNA, bacterial, semisynthetic, or synthetic origin which, by virtue of its origin or manipulation, is not associated with all or a portion of the polynucleotide with which it is associated in nature.

The term “recombinant” as used with respect to a protein or polypeptide means a polypeptide produced by expression of a recombinant polynucleotide. In general, the polynucleotide of interest is cloned and then expressed in transformed host cells, for example, as described herein. The host organism expresses the foreign nucleic acids to produce the RNA, RT-DNA, or protein under expression conditions.

As used herein, a “cell” refers to any type of cell isolated from a prokaryotic, eukaryotic, or archaeon organism, including bacteria, archaea, fungi, protists, plants, and animals, including cells from tissues, organs, and biopsies, as well as recombinant cells, cells from cell lines cultured in vitro, and cellular fragments, cell components, or organelles comprising nucleic acids. The term also encompasses artificial cells, such as nanoparticles, liposomes, polymersomes, or microcapsules encapsulating nucleic acids. The methods described herein can be performed, for example, on a sample comprising a single cell or a population of cells. The term also includes genetically modified cells.

The term “transformation” refers to the insertion of an exogenous polynucleotide (e.g., an engineered retron) into a host cell, irrespective of the method used for the insertion. For example, direct uptake, transduction or f-mating are included. The exogenous polynucleotide may be maintained as a non-integrated vector, for example, a plasmid, or alternatively, may be integrated into the host genome.

“Recombinant host cells,” “host cells”, “cells”, “cell lines”, “cell cultures”, and other such terms denoting microorganisms or higher eukaryotic cell lines cultured as unicellular entities refer to cells which can be, or have been, used as recipients for recombinant vector or other transferred DNA, and include the original progeny of the original cell which has been transfected.

A “coding sequence” or a sequence which “encodes” a selected RNA or polypeptide, is a nucleic acid molecule which is transcribed (in the case of DNA) into RNA and/or translated (in the case of mRNA) into a polypeptide in vivo when placed under the control of appropriate regulatory sequences (or “control elements”). The boundaries of the coding sequence can be determined by a start codon at the 5′ (amino) terminus and a translation stop codon at the 3′ (carboxy) terminus. A coding sequence can include, but is not limited to, cDNA from viral, prokaryotic or eukaryotic ncRNA, mRNA, genomic DNA sequences from retron, viral or prokaryotic DNA, and even synthetic DNA sequences. A transcription termination sequence may be located 3′ to the coding sequence.

Typical “control elements,” include, but are not limited to, transcription promoters, transcription enhancer elements, transcription termination signals, polyadenylation sequences (located 3′ to the translation stop codon), sequences for optimization of initiation of translation (located 5′ to the coding sequence), and translation termination sequences.

“Operably linked” refers to an arrangement of elements wherein the components so described are configured so as to perform their usual function. Thus, a given promoter operably linked to a coding sequence is capable of effecting the expression of the coding sequence when the proper polymerases are present. The promoter need not be contiguous with the coding sequence, so long as it functions to direct the expression thereof. Thus, for example, intervening untranslated yet transcribed sequences can be present between the promoter sequence and the coding sequence and the promoter sequence can still be considered “operably linked” to the coding sequence.

“Encoded by” refers to a nucleic acid sequence which codes for a polypeptide or RNA sequence. For example, the polypeptide sequence or a portion thereof contains an amino acid sequence of at least 3 to 5 amino acids, more preferably at least 8 to 10 amino acids, and even more preferably at least 15 to 20 amino acids from a polypeptide encoded by the nucleic acid sequence. The RNA sequence or a portion thereof contains a nucleotide sequence of at least 3 to 5 nucleotides, more preferably at least 8 to 10 nucleotides, and even more preferably at least 15 to 20 nucleotides.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or RNA or cause other adverse consequences. That is, a nucleic acid or peptide of this invention is purified if it is substantially free of cellular material, viral material, or culture medium when obtained from nature or when produced by recombinant DNA techniques, or free from chemical precursors or other chemicals when chemically synthesized. Purity and homogeneity are typically determined using analytical chemistry techniques, for example, polyacrylamide gel electrophoresis or high-performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

“Substantially purified” generally refers to isolation of a substance (nucleic acid, RNA, DNA, compound, polynucleotide, protein, polypeptide, peptide composition) such that the substance comprises the majority percent of the sample in which it resides. Typically, in a sample, a substantially purified component comprises 50%, preferably 80%-85%, more preferably 90-95% of the sample. Techniques for purifying polynucleotides and polypeptides of interest are available in the art and include, for example, ion-exchange chromatography, affinity chromatography and sedimentation according to density.

“Expression” refers to detectable production of a gene product by a cell. The gene product may be a transcription product (i.e., RNA), which may be referred to as “gene expression”, or the gene product may be a translation product of the transcription product (i.e., a protein), depending on the context. “Purified polynucleotide” refers to a polynucleotide of interest or fragment thereof which is essentially free, e.g., contains less than about 50%, preferably less than about 70%, and more preferably less than about at least 90%, of the protein and/or nucleic acids with which the polynucleotide is naturally associated. Techniques for purifying polynucleotides of interest are available in the art and include, for example, disruption of the cell containing the polynucleotide with a chaotropic agent and separation of the polynucleotide(s) and proteins by ion-exchange chromatography, affinity chromatography and sedimentation according to density.

The term “transfection” is used to refer to the uptake of foreign DNA by a cell. A cell has been “transfected” when exogenous DNA has been introduced inside the cell membrane. A number of transfection techniques are generally available. See, e.g., Graham et al. (1973) Virology, 52:456, Sambrook et al. (2001) Molecular Cloning, a laboratory manual, 3rd edition, Cold Spring Harbor Laboratories, New York, Davis et al. (1995) Basic Methods in Molecular Biology, 2nd edition, McGraw-Hill, and Chu et al. (1981) Gene 13:197. Such techniques can be used to introduce one or more exogenous DNA moieties into suitable host cells. The term refers to both stable and transient uptake of the genetic material and includes uptake of peptide-linked or antibody-linked DNAs.

A “vector” is capable of transferring nucleic acid sequences to target cells (e.g., viral vectors, non-viral vectors, particulate carriers, and liposomes). Typically, “vector construct,” “expression vector,” and “gene transfer vector,” mean any nucleic acid construct capable of directing the expression of a nucleic acid of interest and which can transfer nucleic acid sequences to target cells. Thus, the term includes cloning and expression vehicles, as well as viral vectors.

“Mammalian cell” refers to any cell derived from a mammalian subject suitable for transfection with retron nucleic acids or vector systems comprising retron nucleic acids, as described herein. The cell may be xenogeneic, autologous, or allogeneic. The cell can be a primary cell obtained directly from a mammalian subject. The cell may also be a cell derived from the culture and expansion of a cell obtained from a mammalian subject. Immortalized cells are also included within this definition. In some embodiments, the cell has been genetically engineered to express a recombinant protein and/or nucleic acid.

The term “subject” includes animals, including both vertebrates and invertebrates, including, without limitation, invertebrates such as arthropods, mollusks, annelids, and cnidarians; and vertebrates such as amphibians, including frogs, salamanders, and caecillians; reptiles, including lizards, snakes, turtles, crocodiles, and alligators; fish; mammals, including human and non-human mammals such as non-human primates, including chimpanzees and other apes and monkey species; laboratory animals such as mice, rats, rabbits, hamsters, guinea pigs, and chinchillas; domestic animals such as dogs and cats; farm animals such as sheep, goats, pigs, horses and cows; and birds such as domestic, wild and game birds, including chickens, turkeys and other gallinaceous birds, ducks, geese, and the like. In some cases, the disclosed methods find use in experimental animals, in veterinary application, and in the development of animal models for disease, including, but not limited to, rodents including mice, rats, and hamsters; primates, and transgenic animals.

“Gene transfer” or “gene delivery” refers to methods or systems for reliably inserting DNA or RNA of interest into a host cell. Such methods can result in transient expression of non-integrated transferred DNA, extrachromosomal replication and expression of transferred replicons (e.g., episomes), or integration of transferred genetic material into the genomic DNA of host cells. Gene delivery expression vectors include, but are not limited to, vectors derived from bacterial plasmid vectors, viral vectors, non-viral vectors, alphaviruses, pox viruses and vaccinia viruses.

The term “derived from” is used herein to identify the original source of a molecule but is not meant to limit the method by which the molecule is made which can be, for example, by chemical synthesis or recombinant means.

A polynucleotide or nucleic acid “derived from” a designated sequence refers to a polynucleotide or nucleic acid that includes a contiguous sequence of approximately at least about 6 nucleotides, preferably at least about 8 nucleotides, more preferably at least about 10-12 nucleotides, and even more preferably at least about 15-20 nucleotides corresponding, i.e., identical or complementary to, a region of the designated nucleotide sequence. The derived polynucleotide will not necessarily be derived physically from the nucleotide sequence of interest, but may be generated in any manner, including, but not limited to, chemical synthesis, replication, reverse transcription or transcription, which is based on the information provided by the sequence of bases in the region(s) from which the polynucleotide is derived. As such, it may represent either a sense or an antisense orientation of the original polynucleotide.

A “barcode” refers to one or more nucleotide sequences that are used to identify a nucleic acid or cell with which the barcode is associated. Barcodes can be 3-1000 or more nucleotides in length, preferably 10-250 nucleotides in length, and more preferably 10-30 nucleotides in length, including any length within these ranges, such as 3, 4, 5, 6, 7, 8, 9, 10, 11, 12, 13, 14, 15, 16, 17, 18, 19, 20, 21, 22, 23, 24, 25, 26, 27, 28, 29, 30, 40, 50, 60, 70, 80, 90, 100, 200, 300, 400, 500, 600, 700, 800, 900, or 1000 nucleotides in length. Barcodes may be used, for example, to identify a single cell, subpopulation of cells, colony, or sample from which a nucleic acid originated. Barcodes may also be used to identify the position (i.e., positional barcode) of a cell, colony, or sample from which a nucleic acid originated, such as the position of a colony in a cellular array, the position of a well in a multi-well plate, or the position of a tube, flask, or other container in a rack. For example, a barcode may be used to identify a genetically modified cell from which a nucleic acid originated. In some embodiments, a barcode is used to identify a particular type of genome edit or a particular type of donor nucleic acid.

The terms “hybridize” and “hybridization” refer to the formation of complexes between nucleotide sequences which are sufficiently complementary to form complexes via Watson-Crick base pairing.

The term “homologous region” refers to a region of a nucleic acid with homology to another nucleic acid region. Thus, whether a “homologous region” is present in a nucleic acid molecule is determined with reference to another nucleic acid region in the same or a different molecule. Further, since a nucleic acid is often double-stranded, the term “homologous, region,” as used herein, refers to the ability of nucleic acid molecules to hybridize to each other. For example, a single-stranded nucleic acid molecule can have two homologous regions which are capable of hybridizing to each other. Thus, the term “homologous region” includes nucleic acid segments with complementary sequences. Homologous regions may vary in length but will typically be between 4 and 500 nucleotides (e.g., from about 4 to about 40, from about 40 to about 80, from about 80 to about 120, from about 120 to about 160, from about 160 to about 200, from about 200 to about 240, from about 240 to about 280, from about 280 to about 320, from about 320 to about 360, from about 360 to about 400, from about 400 to about 440, etc.).

As used herein, the terms “complementary” or “complementarity” refers to polynucleotides that are able to form base pairs with one another. Base pairs are typically formed by hydrogen bonds between nucleotide units in an anti-parallel orientation between polynucleotide strands. Complementary polynucleotide strands can base pair in a Watson-Crick manner (e.g., A to T, A to U, C to G), or in any other manner that allows for the formation of duplexes. As persons skilled in the art are aware, when using RNA as opposed to DNA, uracil (U) rather than thymine (T) is the base that is considered to be complementary to adenosine. However, when uracil is denoted in the context of the present invention, the ability to substitute a thymine is implied, unless otherwise stated. “Complementarity” may exist between two RNA strands, two DNA strands, or between an RNA strand and a DNA strand. It is generally understood that two or more polynucleotides may be “complementary” and able to form a duplex despite having less than perfect or less than 100% complementarity. Two sequences are “perfectly complementary” or “100% complementary” if at least a contiguous portion of each polynucleotide sequence, comprising a region of complementarity, perfectly base pairs with the other polynucleotide without any mismatches or interruptions within such region. Two or more sequences are considered “perfectly complementary” or “100% complementary” even if either or both polynucleotides contain additional non-complementary sequences as long as the contiguous region of complementarity within each polynucleotide is able to perfectly hybridize with the other. “Less than perfect” complementarity refers to situations where less than all of the contiguous nucleotides within such region of complementarity are able to base pair with each other. Determining the percentage of complementarity between two polynucleotide sequences is a matter of ordinary skill in the art.

The term “Cas9” as used herein encompasses type II clustered regularly interspaced short palindromic repeats (CRISPR) system Cas9 endonucleases from any species, and also includes biologically active fragments, variants, analogs, and derivatives thereof that retain Cas9 endonuclease activity (i.e., catalyze site-directed cleavage of DNA to generate double-strand breaks). A Cas9 endonuclease binds to and cleaves DNA at a site comprising a sequence complementary to its bound guide RNA (gRNA). For purposes of Cas9 targeting, a gRNA may comprise a sequence “complementary” to a target sequence (e.g., major or minor allele), capable of sufficient base-pairing to form a duplex (i.e., the gRNA hybridizes with the target sequence). Additionally, the gRNA may comprise a sequence complementary to a PAM sequence, wherein the gRNA also hybridizes with the PAM sequence in a target DNA.

The term “donor polynucleotide” refers to a nucleic acid or polynucleotide that provides a sequence of an intended edit to be integrated into the genome at a target locus by HDR or recombineering.

A “target site” or “target sequence” is the nucleic acid sequence recognized (i.e., sufficiently complementary for hybridization) by a guide RNA (gRNA) or a homology arm of a donor polynucleotide. The target site may be allele-specific (e.g., a major or minor allele). For example, a target site can be a genomic site that is intended to be modified such as by insertion of one or more nucleotides, replacement of one or more nucleotides, deletion of one or more nucleotides, or a combination thereof.

By “homology arm” is meant a portion of a donor polynucleotide that is responsible for targeting the donor polynucleotide to the genomic sequence to be edited in a cell. The donor polynucleotide typically comprises a 5′ homology arm that hybridizes to a 5′ genomic target sequence and a 3′ homology arm that hybridizes to a 3′ genomic target sequence flanking a nucleotide sequence comprising the intended edit to the genomic DNA. The homology arms are referred to herein as 5′ and 3′ (i.e., upstream and downstream) homology arms, which relates to the relative position of the homology arms to the nucleotide sequence comprising the intended edit within the donor polynucleotide. The 5′ and 3′ homology arms hybridize to regions within the target locus in the genomic DNA to be modified, which are referred to herein as the “5′ target sequence” and “3′ target sequence,” respectively. For example, the nucleotide sequence comprising the intended edit can be integrated into the genomic DNA by HDR or recombineering at the genomic target locus recognized (i.e., sufficiently complementary for hybridization) by the 5′ and 3′ homology arms.

In general, “a CRISPR system” refers collectively to transcripts and other elements involved in the expression of or directing the activity of CRISPR-associated (“Cas”) genes, including sequences encoding a Cas gene, and a CRISPR array nucleic acid sequence including a leader sequence and at least one repeat sequence. In some embodiments, one or more elements of a CRISPR system are derived from a type I, type II, or type III CRISPR system. Casl and Cas2 are found in all three types of CRISPR-Cas systems, and they are involved in spacer acquisition. In the I-E system of E. coli, Casl and Cas2 form a complex where a Cas2 dimer bridges two Casl dimers. In this complex Cas2 performs a non-enzymatic scaffolding role, binding double-stranded fragments of invading DNA, while Casl binds the single-stranded flanks of the DNA and catalyzes their integration into CRISPR arrays.

In some embodiments, one or more elements of a CRISPR system is derived from a particular organism comprising an endogenous CRISPR system, such as Streptococcus pyogenes. In general, a CRISPR system is characterized by elements that promote the formation of a CRISPR complex at the site of a target sequence (also referred to as a protospacer in the context of an endogenous CRISPR system).

In some embodiments, a vector comprises a regulatory element operably linked to an enzyme-coding sequence encoding a CRISPR enzyme, such as a Cas protein. Non-limiting examples of Cas proteins include Casl, CaslB, Cas2, Cas3, Cas4, Cas5, Cas6, Cas7, Cas8, Cas9 (also known as Csnl and Csxl2), CaslO, Csyl, Csy2, Csy3, Csel, Cse2, Cscl, Csc2, Csa5, Csn2, Csm2, Csm3, Csm4, Csm5, Csm6, Cmrl, Cmr3, Cmr4, Cmr5, Cmr6, Csbl, Csb2, Csb3, Csxl7, Csxl4, CsxlO, Csxl6, CsaX, Csx3, Csxl, Csxl5, Csfl, Csf2, Csf3, Csf4, homologs thereof, or modified versions thereof.

In certain embodiments, the disclosure provides protospacers that are adjacent to short (3-5 bp) DNA sequences termed protospacer adjacent motifs (PAM). The PAMs are important for type I and type II systems during acquisition. In type I and type II systems, protospacers are excised at positions adjacent to a PAM sequence, with the other end of the spacer is cut using a ruler mechanism, thus maintaining the regularity of the spacer size in the CRISPR array. The conservation of the PAM sequence differs between CRISPR-Cas systems and may be evolutionarily linked to Casl and the leader sequence.

In some embodiments, the disclosure provides for integration of defined synthetic DNA that is produced within a cell such as by using an engineered retron system within the cell into a CRISPR array in a directional manner, occurring preferentially, but not exclusively, adjacent to the leader sequence. In the type I-E system from E. coli, it was demonstrated that the first direct repeat, adjacent to the leader sequence is copied, with the newly acquired spacer inserted between the first and second direct repeats.

In one embodiment, the protospacer is a defined synthetic DNA. In some embodiments, the defined synthetic DNA is at least 3, 5, 10, 20, 30, 40, or 50 nucleotides, or between 3-50, or between 10-100, or between 20-90, or between 30-80, or between 40-70, or between 50-60, nucleotides in length. In one embodiment, the oligo nucleotide sequence or the defined synthetic DNA includes a modified “AAG” protospacer adjacent motif (PAM).

In some embodiments, a regulatory element is operably linked to one or more elements of a CRISPR system so as to drive expression of the one or more elements of the CRISPR system. In general, CRISPRs (Clustered Regularly Interspaced Short Palindromic Repeats), also known as SPIDRs (SPacer Interspersed Direct Repeats), constitute a family of DNA loci that are usually specific to a particular bacterial species. The CRISPR locus comprises a distinct class of interspersed short sequence repeats (SSRs) that were recognized in E. coli (Ishino et a1, J. BacterioL, 169:5429-5433 (1987); and Nakata et al., J. BacterioL, 171:3553-3556 (1989)), and associated genes. Similar interspersed SSRs have been identified in Haloferax mediterranei, Streptococcus pyogenes, Anabaena, and Mycobacterium tuberculosis (See, Groenen et al., Mol. Microbiol., 10:1057-1065 (1993); Hoe et al., Emerg. Infect. Dis., 5:254-263 (1999); Masepohl et a1, Biochim. Biophys. Acta 1307:26-30 (1996); and Mojica et a1, Mol. Microbiol, 17:85-93 (1995)). The CRISPR loci typically differ from other SSRs by the structure of the repeats, which have been termed short regularly spaced repeats (SRSRs) (Janssen et a1, OMICS J. Integ. Biol., 6:23-33 (2002); and Mojica et a1, Mol. Microbiol., 36:244-246 (2000)). In general, the repeats are short elements that occur in clusters that are regularly spaced by unique intervening sequences with a substantially constant length (Mojica et al., (2000), supra). Although the repeat sequences are highly conserved between strains, the number of interspersed repeats and the sequences of the spacer regions typically differ from strain to strain (van Embden et al., J. Bacteriol., 182:2393-2401 (2000)). CRISPR loci have been identified in more than 40 prokaryotes (See e.g., Jansen et a1, Mol. Microbiol., 43:1565-1575 (2002); and Mojica et a1, (2005)) including, but not limited to Aeropyrum, Pyrobaculum, Sulfolobus, Archaeoglobus, Halocarcula, Methanobacteriumn, Methanococcus, Methanosarcina, Methanopyrus, Pyrococcus, Picrophilus, Thernioplasnia, Corynebacterium, Mycobacterium, Streptomyces, Aquifrx, Porphvromonas, Chlorobium, Thermus, Bacillus, Listeria, Staphylococcus, Clostridium, Thermoanaerobacter, Mvcoplasma, Fusobacterium, Azarcus, Chromobacterium, Neisseria, Nitrosomonas, Desulfovibrio, Geobacter, Myrococcus, Campylobacter, Wolinella, Acinetobacter, Erwinia, Escherichia, Legionella, Methylococcus, Pasteurella, Photobacterium, Salmonella, Xanthomonas, Yersinia. Treponema, and Thermotoga.

In some embodiments, an enzyme coding sequence encoding a CRISPR enzyme is codon optimized for expression in particular cells, such as eukaryotic cells. The eukaryotic cells may be those of or derived from a particular organism, such as a mammal, including but not limited to human, mouse, rat, rabbit, dog, or non-human primate. In general, codon optimization refers to a process of modifying a nucleic acid sequence for enhanced expression in the host cells of interest by replacing at least one codon (e.g. about one or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database”, and these tables can be adapted in a number of ways. See Nakamura, Y., et al. “Codon usage tabulated from the international DNA sequence databases: status for the year 2000” Nucl. Acids Res. 28:292 (2000). Computer algorithms for codon optimizing a particular sequence for expression in a particular host cell are also available, such as Gene Forge (Aptagen; Jacobus, Pa.), are also available. In some embodiments, one or more codons (e.g. 1, 2, 3, 4, 5, 10, 15, 20, 25, 50, or more, or all codons) in a sequence encoding a CRISPR enzyme correspond to the most frequently used codon for a particular amino acid.

“Administering” a nucleic acid, such as an engineered retron construct or vector comprising an engineered retron construct to a cell comprises transducing, transfecting, electroporating, translocating, fusing, phagocytosing, shooting or ballistic methods, etc., i.e., any means by which a nucleic acid can be transported across a cell membrane.

Before the present disclosure is further described, it is to be understood that the disclosed subject matter is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosed subject matter. The upper and lower limits of these smaller ranges may independently be included in the smaller ranges, and are also encompassed within the disclosed subject matter, subject to any specifically excluded limit in the stated range. Where the stated range includes one or both of the limits, ranges excluding either or both of those included limits are also included in the disclosed subject matter.

Unless defined otherwise, all technical and scientific terms used herein have the same meaning as commonly understood by one of ordinary skill in the art to which the disclosed subject matter belongs. Although any methods and materials similar or equivalent to those described herein can also be used in the practice or testing of the disclosed subject matter, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “a cell” includes a plurality of such cells and reference to “the nucleic acid” includes reference to one or more nucleic acids and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of any features or elements described herein, which includes use of a “negative” limitation.

It is appreciated that certain features of the disclosed subject matter, which are, for clarity, described in the context of separate embodiments, may also be provided in combination in a single embodiment. Conversely, various features of the disclosed subject matter, which are, for brevity, described in the context of a single embodiment, may also be provided separately or in any suitable sub-combination. All combinations of the embodiments pertaining to the disclosure are specifically embraced by the disclosed subject matter and are disclosed herein just as if each and every combination was individually and explicitly disclosed. In addition, all sub-combinations of the various embodiments and elements thereof are also specifically embraced by the present disclosure and are disclosed herein just as if each and every such sub-combination was individually and explicitly disclosed herein.

The publications discussed herein are provided solely for their disclosure prior to the filing date of the present application. Nothing herein is to be construed as an admission that the disclosed subject matter is not entitled to antedate such publication. Further, the dates of publication provided may be different from the actual publication dates which may need to be independently confirmed.

The following Examples illustrate some of the materials, methods, and experiments that were used or performed in the development of the invention.

### Example 1: Sequencing Methods

Retron RNA (ncRNA) and retron reverse transcribed DNA (RT-DNA) synthesis was generated by inducing expression of the ncRNA/reverse transcriptase (RT) in bacteria for five hours, or in yeast and human cells for 24 hours. The RT-DNA was then collected from the cells and purified.

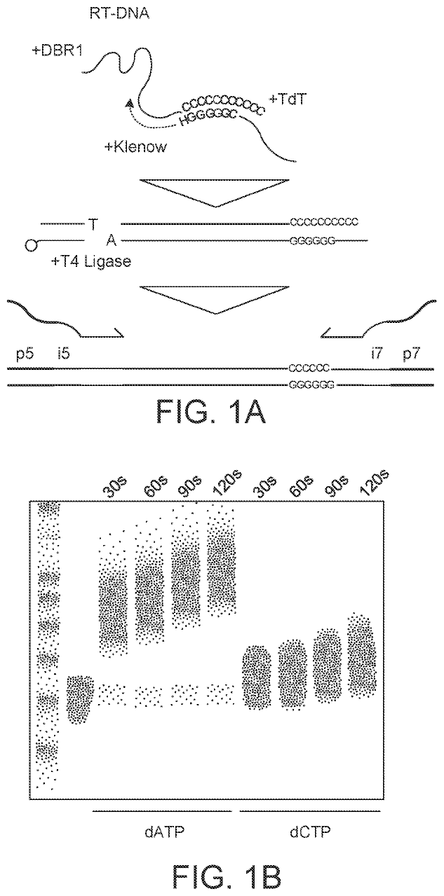

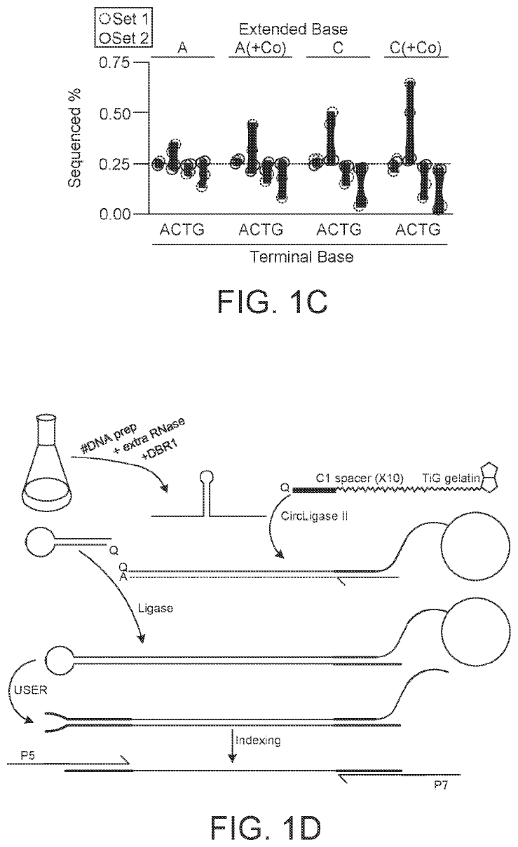

To prepare the RT-DNA for sequencing, the DNA was first treated with DBR1 (OriGene, or inhouse produced DBRT) to remove any RNA branches. The 3′ ends of the RT-DNA were then extended with a single nucleotide, (e.g., dCTP or dATP), in a reaction with terminal deoxynucleotidyl transferase (TdT). This reaction was carried out in the absence of cobalt for 120 seconds at room temperature with the aim of adding only 5-10 cytosines or adenines before inactivating the TdT at 70° C. A second complementary strand was then generated from that extended product using Klenow Fragment (3′→5′ exo⁻) with a primer containing an Illumina adapter sequence, six guanines or thymines, and a non-guanine (H) anchor. Finally, Illumina adapters were ligated on at the 3′ end of the complementary strand using T4 ligase. FIG. 1A illustrates these methods.

In various experiments, dATP, dCTP, or dTTP were used as the extending base, with differences in the extension time and differences in the anchored, complementary primers. Experiments indicated that dATP was the preferred base because it introduced the least bias in sequencing in tests using mixed, known oligonucleotides (FIG. 1B-1C).

In other experiments ssDNA ligation (CircLigase II) of a Biotin-TEG modified oligonucleotide with an adapter was used for purification and preparation of the second strand (FIG. 1D).

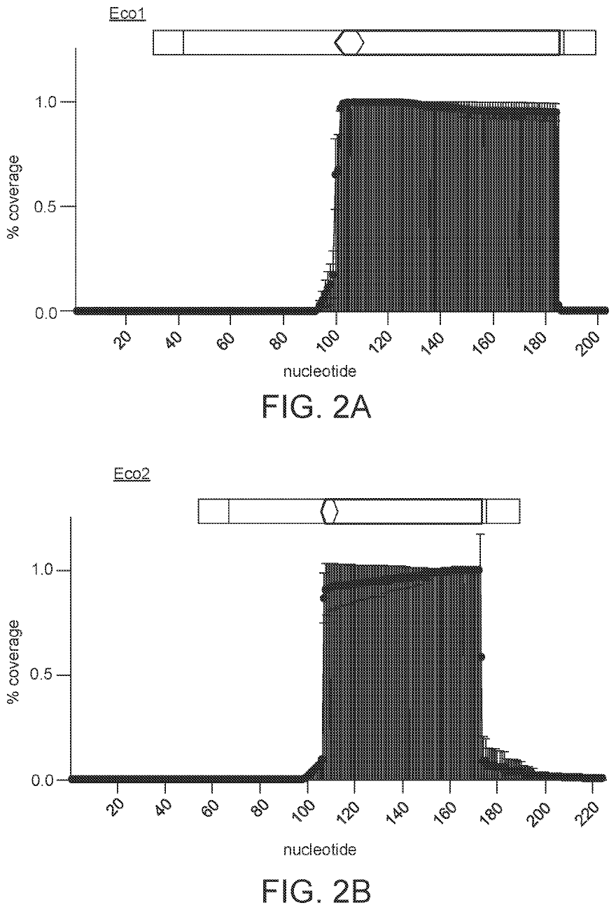

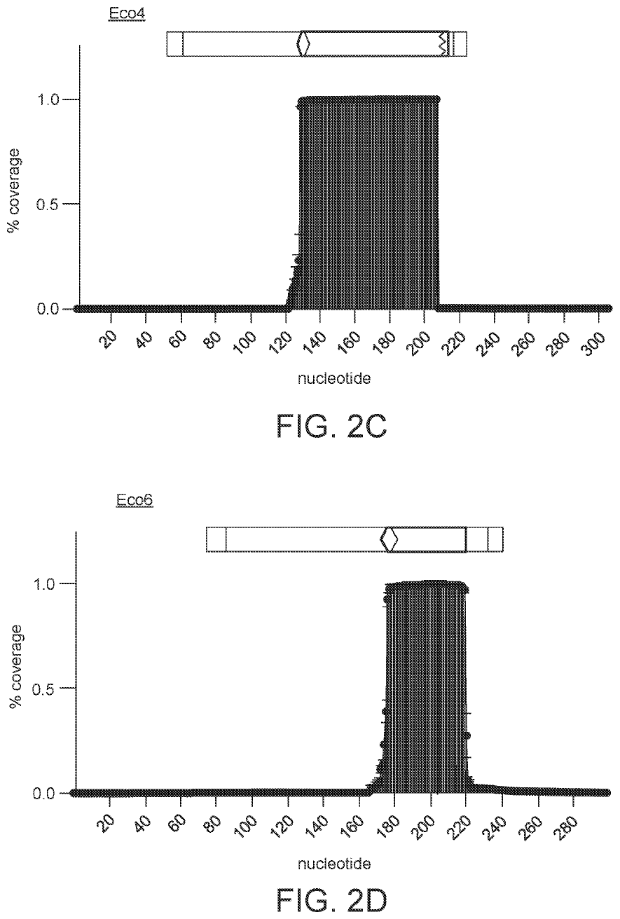

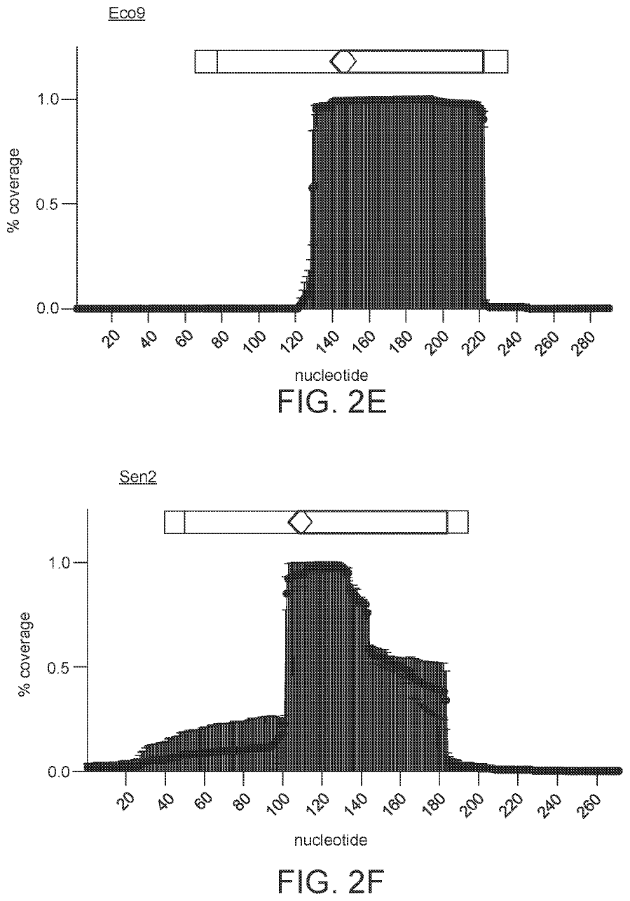

FIG. 2A-2F illustrate the multiplexed sequences observed for six different retron RT-DNAs, where a schematic of the associated ncRNA type is aligned above the RT-DNA sequence, showing the a1/a2 primer region in blue, the ncRNA-RNA portion in pink, and the RT-DNA in brown.

### Example 2: RT-DNA Quantification

This Example illustrates how different RT-DNAs from a library of RT-DNAs can be quantified.

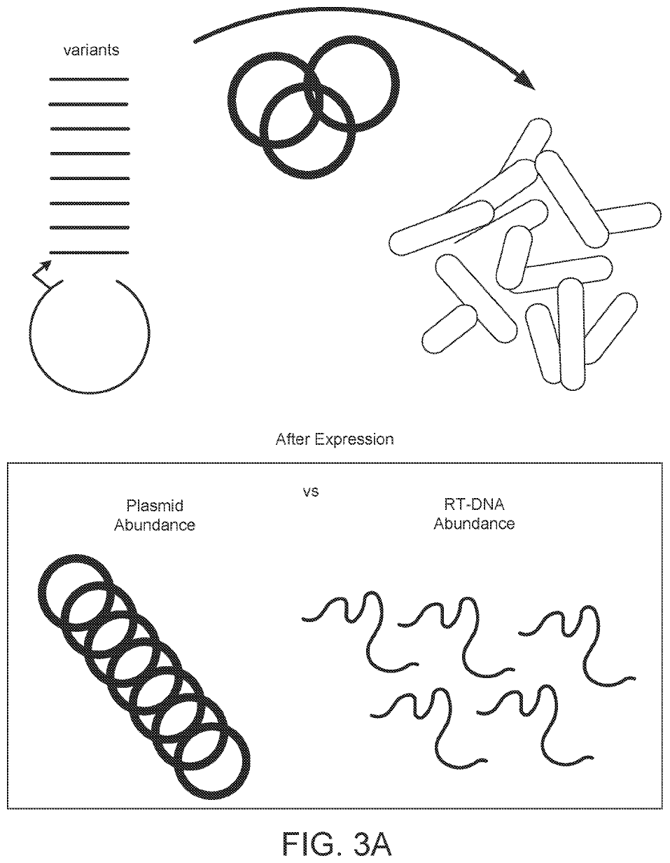

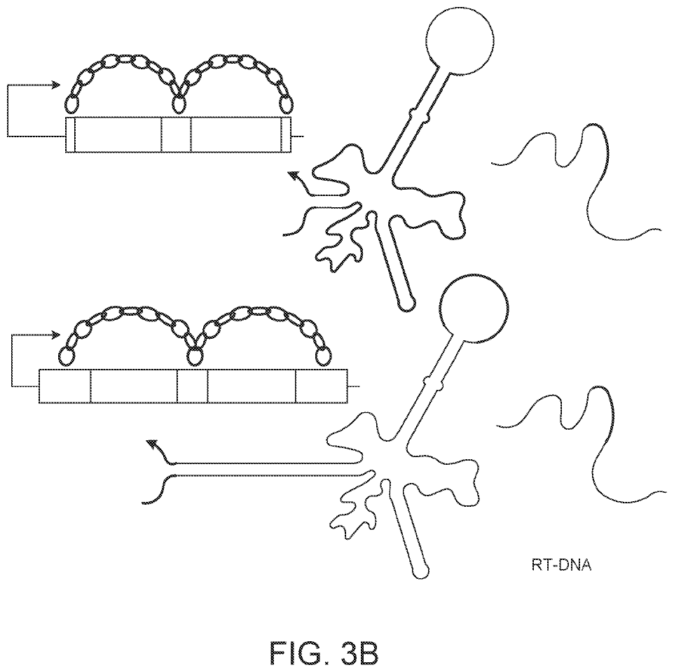

After induction of the ncRNA/RT (5 hours for bacteria, 24 hours for yeast and human cells), DNA as collected from the cells. The abundance of different variant RT-DNAs as well as abundance of different variant plasmids was determined by comparing the relative abundance of a given variant RT-DNA to that variant's plasmid (FIG. 3A). The relative RT-DNA production was estimated, and this RT-DNA production value was normalized to an internal wild-type control for each sub-library.

Quantifying the abundance of different variants required sequencing of RT-DNA using procedures that are not biased by the RT-DNA sequence itself, meaning that different primers in the different RT-DNA could not be used to prepare the RT-DNA for sequencing. Instead, as illustrated in FIG. 1A, a custom RT-DNA sequencing pipeline was developed that involved (1) purifying ssDNA from cells, (2) treating the ssDNA with DBR1 to remove any branched RNA, (3) extending that ssDNA with a single polynucleotide using a template-independent polymerase (TdT), (4) generating a complementary strand using a complementary polynucleotide primer with a single anchor nucleotide and an Illumina adapter, (5) ligating a second adapter to the other end of this double-stranded DNA, and, finally, (6) proceeding to indexing and multiplexed Illumina sequencing.

For any variant modifications that fell outside of the RT-DNA (e.g. regions of the ncRNA that are not reverse transcribed), the mutation(s) in a RT-DNA were cataloged by linking a given mutation to a unique barcode that was introduced into the upper loop of the RT-DNA. Hence, the barcode was associated with only that variant ncRNA/RT-DNA. Such barcodes were sequenced in the RT-DNA pools to quantify variant abundance (FIG. 3B). The relative abundance of the barcoded RT-DNA reflected the effect of the mutation on RT-DNA production.

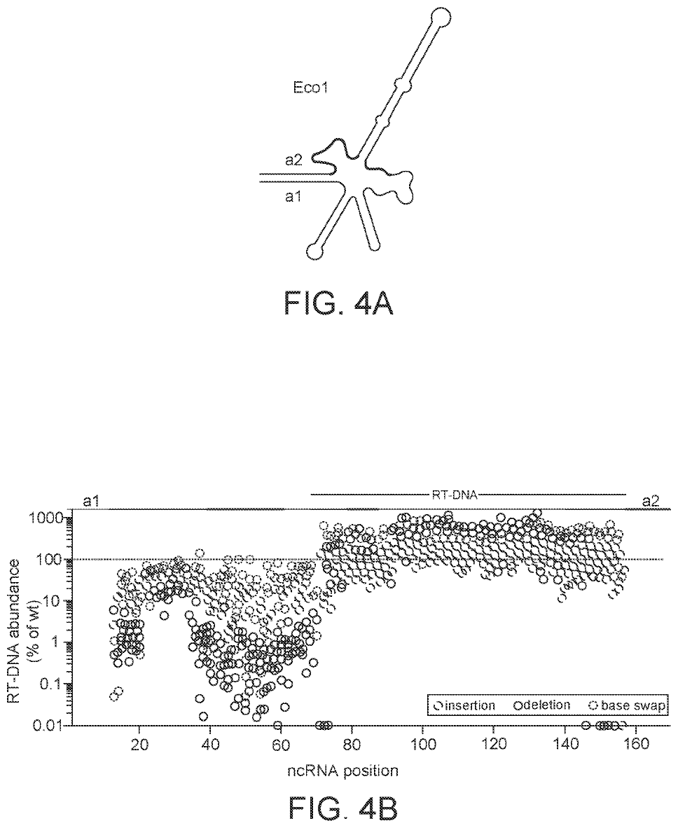

FIG. 4A-4B graphically illustrates quantification of RT-DNA production for 2,158 different insertion, deletion, and base swap mutants relative to their position along a linear ncRNA. Not only does the list of variants continue to produce RT-DNA at or above wild-type levels (FIG. 4B, dotted lines), but FIG. 4B also an understanding of ncRNA regions that are relatively tolerant to mutations (e.g., orange stem loop, blue stem loop in FIG. 4B) and regions that are intolerant (pink stem loop in FIG. 4B).

### Example 3: Evaluating ncRNA/RT-DNA Features

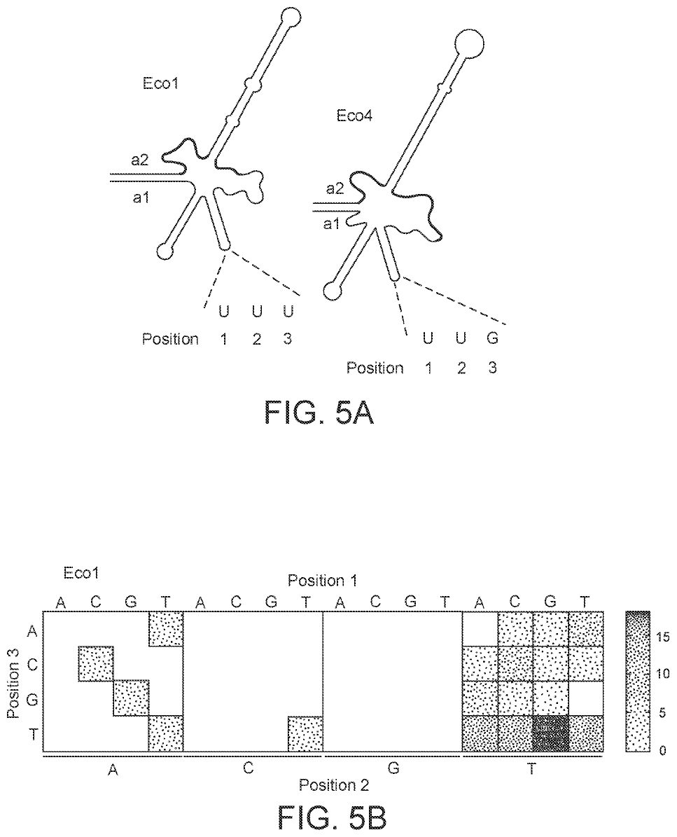

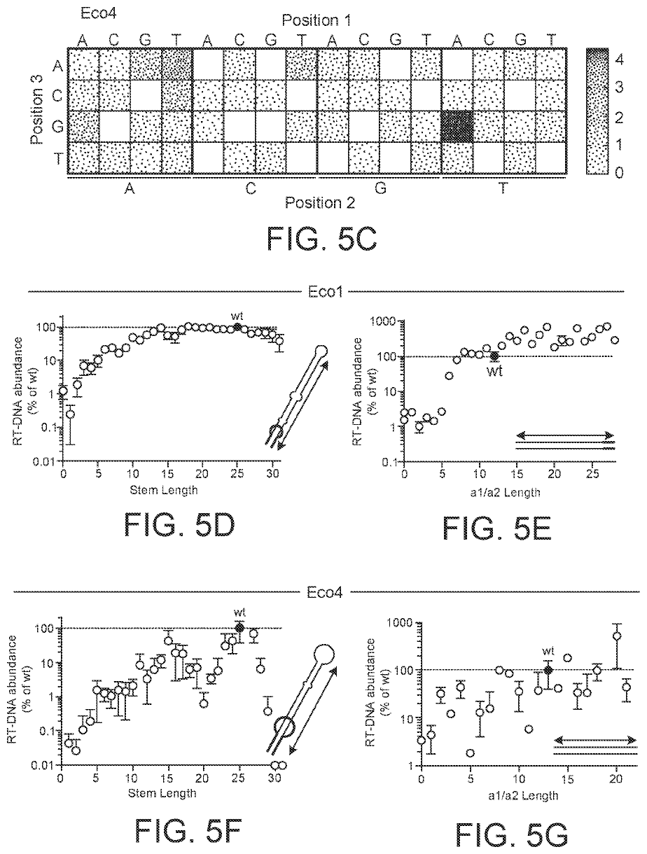

The methods described herein can be used to evaluate features of the ncRNA and to probe similarities and differences between retrons. For example, a region of the ncRNA hypothesized to be involved in reverse transcriptase recognition was determined to be much more sensitive to modifications in Eco1 than Eco4 (FIGS. 5A-5C). In contrast, both Eco1 and Eco4 RT-DNA production is negatively affected by reducing the RT-DNA stem loop length below about 15 base pairs and by reducing the length of the complementary region at the 5′ and 3′ ends of the ncRNA, termed a1/a2, below about 10 base pairs (FIGS. 5D-5G). Interestingly, extension of the a1/a2 region can result in more than a ten-fold increase in RT-DNA production, which is the improvement that can be used to increase editing rates, for example, in yeast.

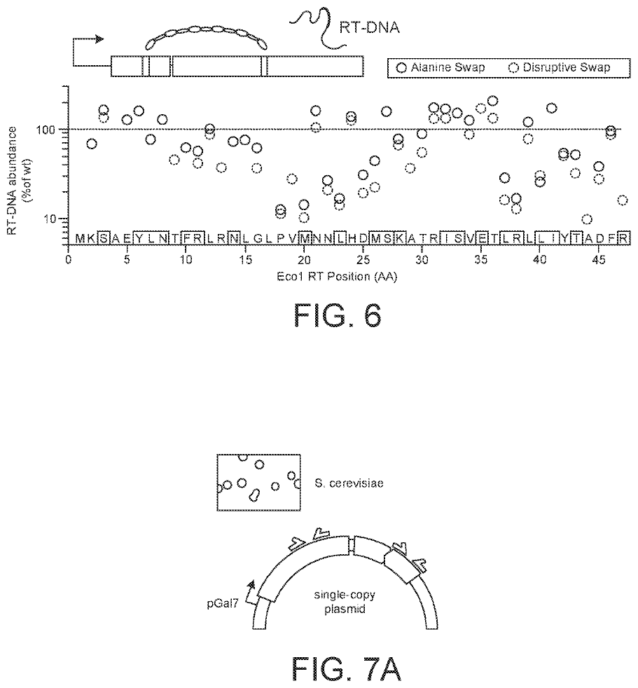

The analytic procedures described herein can also be used to identify and quantify modifications of the protein components of the system, such as the retron reverse transcriptase. To do so, a barcode in the msd region is linked to each modification of the reverse transcriptase gene. Many variant plasmids can be then run in parallel, sequencing the relative abundance of the barcoded RT-DNA to determine the effect of the RT mutation on RT-DNA production (FIG. 6). These experiments were completed in E. coli but can be performed in mammalian cells and yeast.

### Example 4: RT-DNA Production in Eukaryotic Cells

This Example describes experiments designed to evaluate whether increased production by the extended a1/a2 region is a useful modification of retrons expressed and reverse transcribed in eukaryotic cells.

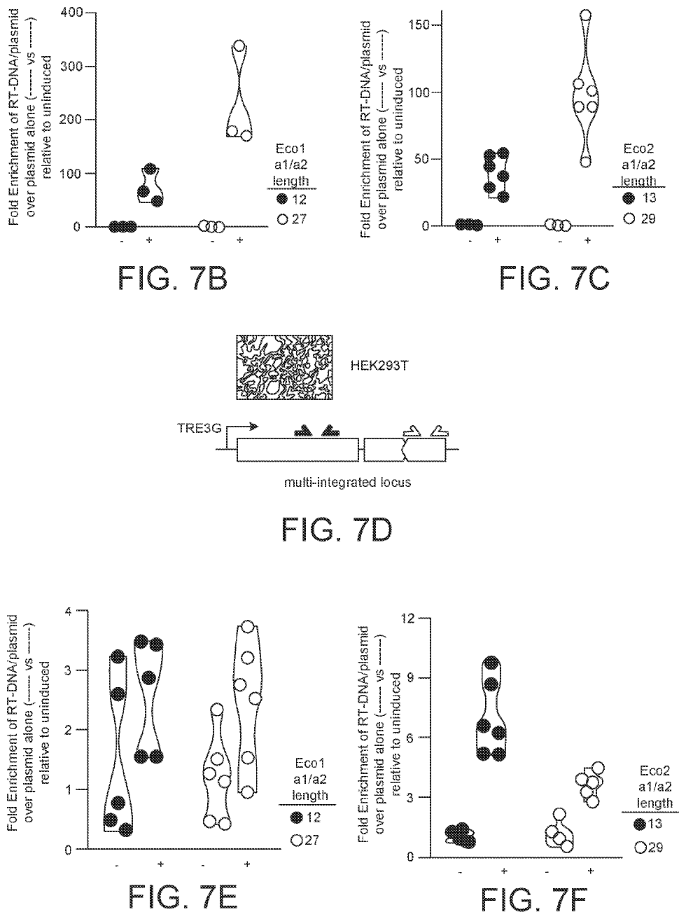

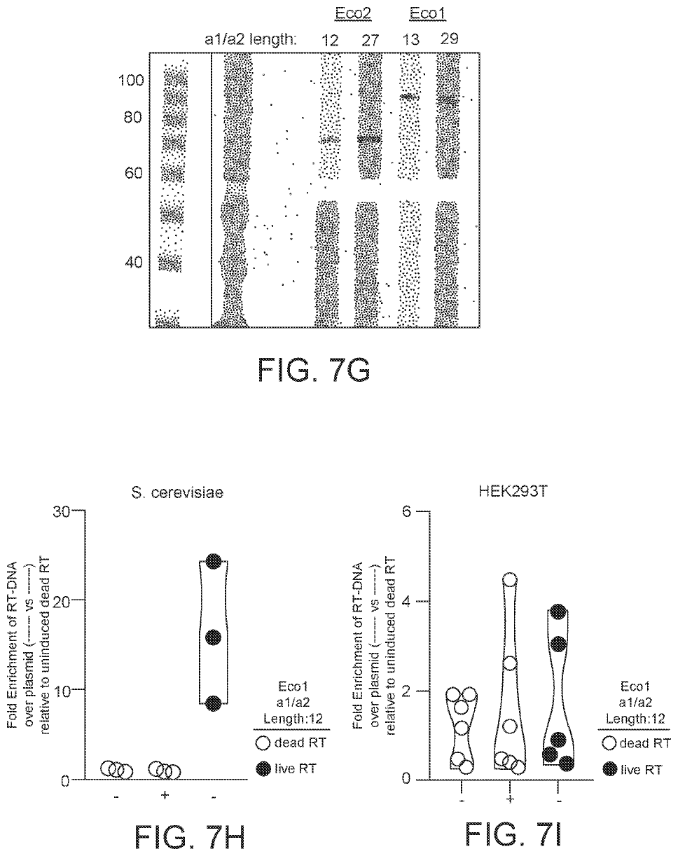

To facilitate expression of Eco1 in eukaryotic cells, the operon was inverted from its native configuration. In the endogenous arrangement, the ncRNA is in the 5′ UTR of the reverse transcriptase transcript, requiring internal ribosome entry for the reverse transcriptase from a ribosomal binding site (RBS) that is in or near the a2 region of the ncRNA. In eukaryotic cells, this arrangement puts the entire ncRNA between the 5′ mRNA cap and the initiation codon for the reverse transcriptase. This increased distance between the cap and initiation codon, as well as the ncRNA structure and out-of-frame ATG codons, can negatively affect reverse transcriptase translation. Moreover, altering the a1/a2 region in the native arrangement could have unintended effects on reverse transcriptase translation. In the inverted architecture designed by the inventors, expression of the reverse transcriptase (gray-shaded in FIG. 7A) is driven by a pol II promoter directly with its initiation codon near the 5′ end of the transcript and the ncRNA in the 3′ UTR, where variations are unlikely to influence reverse transcriptase translation. In FIG. 7A, the segment encoding the reverse transcriptase is shaded grey, while the msd region is blue, and the msr region is pink.

This inverted arrangement was first tested for Eco1 retrons in S. cerevisiae, by placing the RT-ncRNA cassette under the expression of Galactose inducible promoter, on a single-copy plasmid. RT-DNA production was detected using a qPCR assay analogous to that described for E. coli above, comparing amplification from primers that could use the plasmid or RT-DNA as a template to amplification from primers that could anneal only to the plasmid.

As illustrated in FIG. 7B, increasing the length of the Eco1 a1/a2 region from 12 to 27 base pairs resulted in more abundant RT-DNA production in yeast (FIG. 7B, 7G). This analysis was extended to another retron, Eco2. Similar results were obtained: though the wild type ncRNA produced detectable RT-DNA, modified ncRNAs with extended a1/a2 regions ranging from 13 to 29 base pairs produced significantly more RT-DNA in yeast (FIG. 7C, 7G). In each case, induced yeast cells were compared to uninduced cells, which likely under-reports the total RT-DNA abundance if there is any transcriptional ‘leak’ from the plasmid in the absence of inducers. For example, RT-DNA production was detected in the uninduced condition relative to a control expressing a catalytically dead RT, indicating some transcriptional ‘leak’ occurred in uninduced yeast cells (FIG. 7I).

Cultured human cells, HEK293T, were then evaluated for RT-DNA production. The gene architecture used for HEK293T cells was similar to that used for yeast, but with a genome-integrating cassette (FIG. 7D).

As shown in FIG. 7E and FIG. 7I, regardless of a1/a2 length and even when using a tightly regulated promoter, Eco1 did not produce significant abundance of RT-DNA in human cells that was detectable by qPCR. In contrast, Eco2 produced detectable RT-DNA in human cells, with both a wild type and an extended a1/a2 region (FIG. 7F). In contrast to the results in yeast and bacteria, however, the introduction of an extended a1/a2 region diminishes, rather than enhances, production of RT-DNA in human cells. However, RT-DNA production by a retron (e.g., Eco2) can be achieved in human cells when using non-extended (or only slightly extended) a1/a2 regions.

All patents and publications referenced or mentioned herein are indicative of the levels of skill of those skilled in the art to which the invention pertains, and each such referenced patent or publication is hereby specifically incorporated by reference to the same extent as if it had been incorporated by reference in its entirety individually or set forth herein in its entirety. Applicants reserve the right to physically incorporate into this specification any and all materials and information from any such cited patents or publications.

The specific methods and compositions described herein are representative of preferred embodiments and are exemplary and not intended as limitations on the scope of the invention. Other objects, aspects, and embodiments will occur to those skilled in the art upon consideration of this specification and are encompassed within the spirit of the invention as defined by the scope of the claims. It will be readily apparent to one skilled in the art that varying substitutions and modifications may be made to the invention disclosed herein without departing from the scope and spirit of the invention.

The invention illustratively described herein suitably may be practiced in the absence of any element or elements, or limitation or limitations, which is not specifically disclosed herein as essential. The methods and processes illustratively described herein suitably may be practiced in differing orders of steps, and the methods and processes are not necessarily restricted to the orders of steps indicated herein or in the claims.

As used herein and in the appended claims, the singular forms “a,” “an,” and “the” include plural reference unless the context clearly dictates otherwise. Thus, for example, a reference to “a nucleic acid” or “a protein” or “a cell” includes a plurality of such nucleic acids, proteins, or cells (for example, a solution or dried preparation of nucleic acids or expression cassettes, a solution of proteins, or a population of cells), and so forth. In this document, the term “or” is used to refer to a nonexclusive or, such that “A or B” includes “A but not B,” “B but not A,” and “A and B,” unless otherwise indicated.

Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims and statements of the invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein. In addition, where features or aspects of the invention are described in terms of Markush groups, those skilled in the art will recognize that the invention is also thereby described in terms of any individual member or subgroup of members of the Markush group.

1. A method comprising: (a) debranching retron nucleic acids and generating reverse transcribed DNA (RT-DNA) from retron RNA in the retron nucleic acids; (b) extending 3′ ends of the RT-DNA with one type of deoxynucleotide triphosphate (dNTP) in the absence of cobalt to generate 3′-end extended RT-DNA; (c) generating a second strand complement to the 3′-end extended RT-DNA using a DNA polymerase and a primer that binds to the 3′ extended ends of the RT-DNA to generate a double-stranded RT-DNA; (d) attaching adapters onto the 3′ ends of the double-stranded RT-DNA; (e) sequencing the double-stranded RT-DNA; or a combination thereof.

2. The method of claim 1, wherein a debranching enzyme is used for the debranching retron nucleic acids.

3. The method of claim 1, wherein a terminal deoxynucleotidyl transferase (TdT) is used for the extending 3′ ends of the RT-DNA.

4. (canceled)

5. (canceled)

6. (canceled)

7. (canceled)

8. (canceled)

9. The method of claim 1, wherein the one type of deoxynucleotide triphosphate (dNTP) is dATP, dCTP, dGTP, or dTTP.

10. (canceled)

11. (canceled)

12. (canceled)

13. The method of claim 1, wherein attaching adapters onto the 3′ ends of the double-stranded RT-DNA is carried out using a ligase.

14. (canceled)

15. The method of claim 13, wherein the ligase is a T4 ligase or a ligase that can catalyze ligation between single-stranded DNA (ssDNA) and/or single-stranded RNA (ssRNA) substrates.

16. The method of claim 1, wherein the adapters comprise oligonucleotides.

17. (canceled)

18. The method of claim 1, wherein the second strand complement is made using a DNA polymerase without 3′→5′ exonuclease.

19. The method of claim 1, wherein the retron nucleic acids are single-stranded.

20. The method of claim 1, wherein the retron nucleic acids are a mix of single-stranded nucleic acids and double-stranded nucleic acids.

21. The method of claim 1, wherein the retron nucleic acids are oligonucleotides.

22. (canceled)

23. The method of claim 1, wherein the retron nucleic acids are of one type of retron.

24. The method of claim 1, wherein the retron nucleic acids comprise at least one library of retron nucleic acids.

25. The method of claim 24, wherein the at least one library of retron nucleic acids comprise one or more libraries of retron ncRNA variants, retron ncRNA mutants, engineered retron ncRNAs, reverse transcriptases, reverse transcriptase variants, reverse transcriptase mutants, engineered reverse transcriptases, or a combination thereof.

26. The method of claim 24, wherein the at least one library of retron nucleic acids comprise retron nucleic acids linked to one or more exogenous or heterologous RNA or DNA.

27. The method of claim 26, wherein the exogenous or heterologous RNA or DNA comprises or codes for one or more gRNAs, DNA templates for genetic repair, antigens, proteins, regulatory RNAs of interest, or a combination thereof.

28. The method of claim 26, wherein the exogenous or heterologous RNA or DNA is located in a loop region of the retron RNA (retron ncRNA).

29. The method of claim 1, furthering comprising quantifying amounts of each type of double-stranded RT-DNA having a distinct sequence.

30. An expression vector comprising in a 5′ to 3′ direction a first nucleotide sequence that encodes a retron RT RNA transcript from a first promoter and a second nucleotide sequence that encodes a modified retron ncRNA from a second promoter, wherein modified retron ncRNA comprises an extended a1/a2 region.

31. The expression vector of claim 30, wherein the a1/a2 region of the ncRNA is between 13 to 29 base pairs.
